(19) United States
(12) Patent Application Publication
(10) Pub. No.: US 2026/0229136 A1
(43) Pub. Date: Aug. 6, 2026

(54) METHODS AND DEVICES FOR INDUCING NEURAL STIMULATION

(71) Applicant: BAR-ILAN UNIVERSITY, Ramat Gan (IL)

(72) Inventors: Uri POLAT, Ramat Gan (IL); Maria LEV, Herzliya (IL); Ziv SIMANTOV, Haifa (IL); Oren KADOSH, Ramat Gan (IL); Shai NOVIK, Ramat Hasharon (IL)

(73) Assignee: Bar-Ilan University, Ramat Gan (IL)

(21) Appl. No.: 19/150,194

(22) PCT Filed: Jan. 25, 2024
(86) PCT No.: PCT/IL2024/050102

§ 371 Date: Jul. 23, 2025

Related U.S. Application Data
(60) Provisional application No. 63/441,187, filed on Jan. 26, 2023.

(51) Int. Cl. G09B 5/02; G16H 20/70

(52) U.S. Cl. CPC G09B 5/02; G16H 20/70

(57) ABSTRACT

A device and methods thereof to for increasing a user's cognitive abilities, via presenting to the user one or more visual training-tasks, via processor implemented method steps of displaying, at least one session of KS visual training-tasks, requiring the user's one or more responses; whereby the visual training-tasks are configured to induce an amplification of the brain's frequency power, in reaction to at least the provided visual training-tasks, thereby increasing at least one of the user's cognitive abilities.

### BACKGROUND OF THE INVENTION

Network activity within the brain in the gamma frequency (30-100 Hz) plays an important role in information transfer across connected brain regions and across cortical hemispheres. Such oscillatory activity brings multimodal inputs together in a target region for efficient spatio-temporal integration. The gamma-frequency oscillation was shown to slow down lose power in mouse model of Alzheimer's Disease. Transcranial alternating current stimulation projecting Gamma waves was shown to positively affect the long-lasting enhancement of synaptic transmission in mice models of Alzheimer's Disease.

### SUMMARY OF THE INVENTION

According to various embodiments of the invention, new methods are provided for improving and/or increasing a user's cognitive abilities.

According to some embodiments of the invention, a new method is provided for increasing a user's cognitive abilities, the method comprising presenting to the user one or more visual training-tasks, via processor implemented method steps of displaying, at least one session (S₁, S₂ . . . S_{M}), each session of K_{S} visual training-tasks, requiring the user's one or more responses; wherein the visual training-tasks are configured to induce an amplification of the brain's frequency power, in reaction to at least the provided visual training-tasks.

According to some embodiments, the method further comprising:
receiving and/or collecting, at a given time-step N (N=K, 1≤K≤K_{S}) of a session S₁ of K_{S} time steps, the user's response-profile to a displayed visual training-task, displayed at said given time step (N=K);
analyzing the user's response-profile, received at said given time-step (N=K) and optionally any former time-step/s (N<K) of said session S₁;
repeating the step of displaying, at following time-step/s (K=K+1) of said session S₁, a predetermined number of times K≤K_{S} for said session S₁ and/or until the response-profile has reached a predetermined threshold.

According to some embodiments, the method further comprising:
receiving and/or collecting, at a last time-step (N=K_{S}) of a session S₁ of time-steps (N=K, 1≤K≤K_{S}), the user's response-profile to a plurality of displayed visual training-tasks, displayed at time steps N (N=K, 1≤K≤K_{S}) of session S₁;
analyzing the user's response-profile, received at said last time-step (N=K_{S}) of session S₁;
repeating the steps of displaying, at following time-steps N (N=K, 1≤K≤K_{S}) of a following session S₂, a predetermined number of sessions and/or until the response-profile has reached a predetermined threshold.

According to some embodiments, inducement of the amplification of the brain's frequency power, comprises increasing of the power of the Gamma wave.

According to some embodiments, inducement of the amplification of the brain's frequency power, comprises at least one of:
amplification of a P300 positive brainwave component; and
shortening of a P300 latency of the brain's reaction, after the user's respond to the provided visual training-task.

According to some embodiments, the visual training-task/s is/are selected to induce an amplification of the brain's frequency power in reaction at least one visual test-task and/or test-stimulation, which was/were not provided to said user by said method.

According to some embodiments, the visual task comprises differentiating between displays of target and non-target images.

According to some embodiments, the induced amplification of the brain's frequency power, in reaction to the provided visual training-tasks is configured for improving a condition of a patient suffering from at least one of: sub-normal cognitive response, sub-normal motoric response, Alzheimer, depression, schizophrenia, ADHD, dyslexia, Parkinson, multiple sclerosis and any combination thereof.

According to some embodiments, the method further comprising a use in the treatment of at least one of: sub-normal cognitive response, sub-normal motoric response, Alzheimer, depression, schizophrenia, ADHD, dyslexia, Parkinson, multiple sclerosis and any combination thereof.

According to some embodiments of the invention, a new method is provided for increasing a user's brain coherence between visual and cognitive regions, the method comprising presenting one or more visual training-tasks via processor implemented method steps of displaying, at least one session (S₁, S₂ . . . S_{M}) each of K_{S} visual training-tasks to the user; wherein the visual training-tasks are configured to induce an increase in the power of the brain's Gamma wave at local regions, in reaction to at least the provided visual training-tasks.

According to some embodiments, the method further comprising:
receiving and/or collecting, at a given time-step N (N=K, 1≤K≤K_{S}) of a session S₁ of K_{S} time steps, the user's response-profile to a displayed visual training-task, displayed at said given time step (N=K);
analyzing the user's response-profile, received at said given time-step (N=K) and optionally any former time-step/s (N<K) of said session S₁;
repeating the step of displaying, at following time-step/s (K=K+1) of said session S₁, a predetermined number of times K≤K_{S} for said session S₁ and/or until the response-profile has reached a predetermined threshold.

According to some embodiments, the method further comprising:
receiving and/or collecting, at a last time-step (N=K_{S}) of a session S₁ of time-steps (N=K, 1≤K≤K_{S}), the user's response-profile to a plurality of displayed visual training-tasks, displayed at time steps N (N=K, 1≤K≤K_{S}) of session S₁;
analyzing the user's response-profile, received at said last time-step (N=K_{S}) of session S₁;
repeating the steps of displaying, at following time-steps N (N=K, 1≤K≤K_{S}) of a following session S₂, a predetermined number of sessions and/or until the response-profile has reached a predetermined threshold.

According to some embodiments, the visual training-tasks are further configured to induce at least one of:
amplification of a P300 positive brainwave component; and
shortening of a P300 latency of the brain's reaction, after the user's respond to the provided visual training-task.

According to some embodiments, the visual training-task/s is/are selected to induce an amplification of the brain's frequency power in reaction at least one visual test-task and/or test-stimulation, which was/were not provided to said user by said method.

According to some embodiments, the induced amplification of the brain's frequency power, in reaction to the provided visual training-tasks is configured for improving a condition of a patient suffering from at least one of: sub-normal cognitive response, sub-normal motoric response, Alzheimer, depression, schizophrenia, ADHD, dyslexia, Parkinson, multiple sclerosis and any combination thereof.

According to some embodiments, the method further comprising a use in the treatment of at least one of: sub-normal cognitive response, sub-normal motoric response, Alzheimer, depression, schizophrenia, ADHD, dyslexia, Parkinson, multiple sclerosis, and any combination thereof.

According to some embodiments of the invention, a new method is provided for increasing a user's cognitive abilities, the method comprising presenting to the user one or more training stimulating-images, via processor implemented method steps of displaying, at least one session (S₁, S₂ . . . S_{M}) each of K_{S} training stimulating-images; wherein the training stimulating-images are configured to induce an amplification of the brain's frequency power, in reaction to at least to the provided training stimulating-images.

According to some embodiments, inducement of the amplification of the brain's frequency power, comprises increasing of the power of the Gamma wave.

According to some embodiments, inducement of the amplification of the brain's frequency power, comprises at least one of:
amplification of a P300 positive brainwave component; and
shortening of a P300 latency of the brain's reaction, after the provided training stimulating-image.

According to some embodiments, the training stimulating-image/s is/are selected to induce an amplification of the brain's frequency power in reaction at least test-image and/or test-visual-stimulation, which was/were not provided to said user by said method.

According to some embodiments, the induced amplification of the brain's frequency power, in reaction to the provided visual-stimulation is configured for improving a condition of a patient suffering from at least one of: sub-normal cognitive response, sub-normal motoric response, Alzheimer, depression, schizophrenia, ADHD, dyslexia, Parkinson, multiple sclerosis and any combination thereof.

According to some embodiments, the method further comprising a use in the treatment of at least one of: sub-normal cognitive response, sub-normal motoric response, Alzheimer, depression, schizophrenia, ADHD, dyslexia, Parkinson, multiple sclerosis and any combination thereof.

According to some embodiments of the invention, a new method is provided for increasing a user's brain coherence between visual and cognitive regions, the method comprising presenting one or more training stimulating-images via processor implemented method steps of displaying, at least one session (S₁, S₂ . . . S_{M}) each of K_{S} visual-stimulations to the user; wherein the training stimulation-images are configured to induce an increase in the power the brain's Gamma wave at local regions, in reaction to at least the provided training stimulating-images.

According to some embodiments, the training stimulating-images are further configured to induce at least one of:
amplification of a P300 positive brainwave component; and
shortening of a P300 latency of the brain's reaction, after the user is exposed to the provided visual training-stimulation.

According to some embodiments, the visual training-stimulation/s is/are selected to induce an amplification of the brain's frequency power in reaction at least one test-image and/or test-visual-stimulation, which was not provided to said user by said method.

According to some embodiments, the induced amplification of the brain's frequency power, in reaction to the provided training stimulating images is configured for improving a condition of a patient suffering from at least one of: sub-normal cognitive response, sub-normal motoric response, Alzheimer, depression, schizophrenia, ADHD, dyslexia, Parkinson, multiple sclerosis and any combination thereof.

According to some embodiments, the method further comprising a use in the treatment of at least one of: sub-normal cognitive response, sub-normal motoric response, Alzheimer, depression, schizophrenia, ADHD, dyslexia, Parkinson, multiple sclerosis and any combination thereof.

According to some embodiments of the invention a new method is provided for increasing a user's cognitive abilities, the method comprising presenting to the user one or more visual training-tasks, via processor implemented method steps of displaying, at least one session (S₁, S₂ . . . ), each session of K_{S} visual training-tasks, requiring the user's one or more responses; whereby the visual training-tasks are configured to induce a long-term amplification of the brain's frequency power, in reaction to at least the provided visual training-tasks, thereby increasing at least one of the user's cognitive abilities.

According to some embodiments, the method further comprising:
receiving and/or collecting, at a given time-step N (N=K, 1≤K≤K_{S}) of a session S₁ of K_{S} time steps, the user's response-profile to a displayed visual training-task, displayed at the given time step (N=K);
analyzing the user's response-profile, received at the given time-step (N=K) and optionally any former time-step/s (N<K) of the session S₁;
repeating the step of displaying, at following time-step/s (K=K+1) of the session S₁, a predetermined number of times K<K_{S} for the session S₁ and/or until the response-profile has reached a predetermined threshold.

According to some embodiments, the method further comprising:
receiving and/or collecting, at a last time-step (N=K_{S}) of a session S₁ of time-steps (N=K, 1≤K≤K_{S}), the user's response-profile to a plurality of displayed visual training-tasks, displayed at time steps N (N=K, 1≤K≤K_{S}) of session S₁;
analyzing the user's response-profile, received at the last time-step (N=K_{S}) of session S₁;
repeating the steps of displaying, at following time-steps N (N=K, 1≤K≤K_{S}) of a following session S₂, a predetermined number of sessions and/or until the response-profile has reached a predetermined threshold.

According to some embodiments, inducement of the amplification of the brain's frequency power, comprises increasing of the power of the Gamma wave.

According to some embodiments, inducement of the amplification of the brain's frequency power, comprises at least one of:
amplification of a P300 positive brainwave component; and
shortening of a P300 latency of the brain's reaction, after the user's respond to the provided visual training-task.

According to some embodiments, the visual training-task/s is/are selected to induce a long-term amplification of the brain's frequency power in reaction at least one visual test-task and/or test stimulation, which was not provided to the user by the method

According to some embodiments, the visual task comprises differentiating between displays of target and non-target images.

According to some embodiments, the induced long-term amplification of the brain's frequency power, in reaction to the provided visual training-tasks is configured for improving a condition of a patient suffering from at least one of: sub-normal cognitive response, sub-normal motoric response, Alzheimer, depression, schizophrenia, ADHD, dyslexia, Parkinson, multiple sclerosis and any combination thereof.

According to some embodiments, the method further comprising a use in the treatment of at least one of: sub-normal cognitive response, sub-normal motoric response, Alzheimer, depression, schizophrenia, ADHD, dyslexia, Parkinson, multiple sclerosis and any combination thereof.

According to some embodiments of the invention a new method is provided for increasing the power of a user's brain's Gamma wave, the method comprising presenting one or more visual training-tasks via processor implemented method steps of displaying, at least one session S (S₁ . . . S_{M}) each of K_{S} visual training-tasks to the user; whereby the visual training-tasks are configured to induce a long-term increase in the power the brain's Gamma wave, in reaction to at least the provided visual training-tasks.

According to some embodiments, the method further comprising:
receiving and/or collecting, at a given time-step N (N=K, 1≤K≤K_{S}) of a session S₁ of K_{S} time steps, the user's response-profile to a displayed visual training-task, displayed at the given time step (N=K);
analyzing the user's response-profile, received at the given time-step (N=K) and optionally any former time-step/s (N<K) of the session S₁;
repeating the step of displaying, at following time-step/s (K=K+1) of the session S₁, a predetermined number of times K≤K_{S} for the session S₁ and/or until the response-profile has reached a predetermined threshold.

According to some embodiments, the method further comprising:
receiving and/or collecting, at a last time-step (N=K_{S}) of a session S₁ of time-steps (N=K, 1≤K≤K_{S}), the user's response-profile to a plurality of displayed visual training-tasks, displayed at time steps N (N=K, 1≤K≤K_{S}) of session S₁;
analyzing the user's response-profile, received at the last time-step (N=K_{S}) of session S₁;
repeating the steps of displaying, at following time-steps N (N=K, 1≤K≤K_{S}) of a following session S₂, a predetermined number of sessions and/or until the response-profile has reached a predetermined threshold.

According to some embodiments, the visual training-tasks are further configured to induce at least one of:
amplification of a P300 positive brainwave component; and
shortening of a P300 latency of the brain's reaction, after the user's respond to the provided visual training-task.

According to some embodiments, the visual training-task/s is/are selected to induce a long-term amplification of the brain's frequency power in reaction at least one visual test-task and/or test-stimulation, which was not provided to the user by the method.

According to some embodiments, the induced long-term amplification of the brain's frequency power, in reaction to the provided visual training-tasks is configured for improving a condition of a patient suffering from at least one of: sub-normal cognitive response, sub-normal motoric response, Alzheimer, depression, schizophrenia, ADHD, dyslexia, Parkinson, multiple sclerosis and any combination thereof.

According to some embodiments, the method further comprising a use in the treatment of at least one of: sub-normal cognitive response, sub-normal motoric response, Alzheimer, depression, schizophrenia, ADHD, dyslexia, Parkinson, multiple sclerosis and any combination thereof.

According to some embodiments of the invention a new method is provided for amplifying of a P300 positive brainwave component; and/or shortening of the P300 latency of a user's brain's wave, the method comprising presenting one or more visual training-tasks via processor implemented method steps of displaying, at least one session of K_{S} visual training-tasks to the user; whereby the visual training-tasks are configured to induce a long term: amplification of a P300 positive brainwave component; and/or shortening of the P300 latency of the brain's reaction, in reaction to at least the provided visual training-tasks.

According to some embodiments, the method further comprising:
receiving and/or collecting, at a given time-step N (N=K, 1≤K≤K_{S}) of a session S₁ of K_{S} time steps, the user's response-profile to a displayed visual training-task, displayed at the given time step (N=K);
analyzing the user's response-profile, received at the given time-step (N=K) and optionally any former time-step/s (N<K) of the session S₁;
repeating the step of displaying, at following time-step/s (K=K+1) of the session S₁, a predetermined number of times K≤K_{S} for the session S₁ and/or until the response-profile has reached a predetermined threshold.

According to some embodiments, the method further comprising:
receiving and/or collecting, at a last time-step (N=K_{S}) of a session S₁ of time-steps (N=K, 1≤K≤K_{S}), the user's response-profile to a plurality of displayed visual training-tasks, displayed at time steps N (N=K, 1≤K≤K_{S}) of session S₁;
analyzing the user's response-profile, received at the last time-step (N=K_{S}) of session S₁;
repeating the steps of displaying, at following time-steps N (N=K, 1≤K≤K_{S}) of a following session S₂, a predetermined number of sessions and/or until the response-profile has reached a predetermined threshold.

According to some embodiments, training-tasks are further configured to induce an increase to the power of the Gamma wave.

According to some embodiments, wherein the visual training-task/s is/are selected to induce a long-term amplification of the brain's frequency power in reaction at least one visual test-task and/or test-stimulation, which was not provided to the user by the method.

According to some embodiments, the induced long-term amplification of the brain's frequency power, in reaction to the provided visual training-tasks is configured for improving a condition of a patient suffering from at least one of: sub-normal cognitive response, sub-normal motoric response, Alzheimer, depression, schizophrenia, ADHD, dyslexia, Parkinson, multiple sclerosis and any combination thereof.

According to some embodiments, the method further comprising a use in the treatment of at least one of: sub-normal cognitive response, sub-normal motoric response, Alzheimer, depression, schizophrenia, ADHD, dyslexia, Parkinson, multiple sclerosis and any combination thereof.

According to some embodiments of the invention a new method is provided for increasing a user's cognitive abilities, the method comprising presenting to the user one or more training stimulating images via processor implemented method steps of displaying, at least one session S (S₁ . . . S_{M}) of K_{S} stimulating images; wherein the training stimulating images are configured to induce a long-term amplification of the brain's frequency power, in reaction to at least to the provided visual training-stimulation/s.

According to some embodiments, inducement of the amplification of the brain's frequency power, comprises increasing of the power of the Gamma wave.

According to some embodiments, inducement of the amplification of the brain's frequency power, comprises at least one of:
amplification of a P300 positive brainwave component; and
shortening of a P300 latency of the brain's reaction, after the provided visual training-stimulation.

According to some embodiments, the training stimulating images is/are selected to induce a long-term amplification of the brain's frequency power in reaction at least one visual-stimulation and/or test-task, which was not provided to the user by the method.

According to some embodiments, the induced long-term amplification of the brain's frequency power, in reaction to the provided visual-stimulation is configured for improving a condition of a patient suffering from at least one of: sub-normal cognitive response, sub-normal motoric response, Alzheimer, depression, schizophrenia, ADHD, dyslexia, Parkinson, multiple sclerosis and any combination thereof.

According to some embodiments, the method further comprising a use in the treatment of at least one of: sub-normal cognitive response, sub-normal motoric response, Alzheimer, depression, schizophrenia, ADHD, dyslexia, Parkinson, multiple sclerosis and any combination thereof.

According to some embodiments of the invention a new method is provided for increasing the power a user's brain's Gamma wave, the method comprising presenting one or more training stimulating images via processor implemented method steps of displaying, at least one session S (S₁ . . . S_{M}) each of K_{S} visual-stimulations to the user; whereby the visual training-stimulation are configured to induce a long-term increase in the power the brain's Gamma wave, in reaction to at least the provided training stimulating images.

According to some embodiments, the training stimulating images are further configured to induce at least one of:
amplification of a P300 positive brainwave component; and
shortening of a P300 latency of the brain's reaction, after the user is exposed to the provided visual training-stimulation.

According to some embodiments, the visual training-stimulation/s is/are selected to induce a long-term amplification of the brain's frequency power in reaction at least one visual test-stimulation and/or test-task, which was not provided to the user by the method.

According to some embodiments, the induced long-term amplification of the brain's frequency power, in reaction to the provided training stimulating images is configured for improving a condition of a patient suffering from at least one of: sub-normal cognitive response, sub-normal motoric response, Alzheimer, depression, schizophrenia, ADHD, dyslexia, Parkinson, multiple sclerosis and any combination thereof.

According to some embodiments, the method further comprising a use in the treatment of at least one of: sub-normal cognitive response, sub-normal motoric response, Alzheimer, depression, schizophrenia, ADHD, dyslexia, Parkinson, multiple sclerosis and any combination thereof.

According to some embodiments of the invention a new method is provided for amplifying of a P300 positive brainwave component; and/or shortening of the P300 latency of a user's brain's wave, the method comprising presenting one or more training stimulating images via processor implemented method steps of displaying, at least one session of K_{S} training stimulating images to the user;

whereby the training stimulating images are configured to induce a long term: amplification of a P300 positive brainwave component; and/or shortening of the P300 latency of the brain's reaction, in reaction to at least the provided training stimulating images.

According to some embodiments, training-stimulations are further configured to induce an increase to the power of the Gamma wave.

According to some embodiments, the visual training-stimulation/s is/are selected to induce a long-term amplification of the brain's frequency power in reaction at least one visual test-stimulation and/or test-task, which was not provided to the user by the method.

According to some embodiments, the induced long-term amplification of the brain's frequency power, in reaction to the provided training stimulating images is configured for improving a condition of a patient suffering from at least one of: sub-normal cognitive response, sub-normal motoric response, Alzheimer, depression, schizophrenia, ADHD, dyslexia, Parkinson, multiple sclerosis and any combination thereof.

According to some embodiments, the method further comprising a use in the treatment of at least one of: sub-normal cognitive response, sub-normal motoric response, Alzheimer, depression, schizophrenia, ADHD, dyslexia, Parkinson, multiple sclerosis and any combination thereof.

According to some embodiments of the invention a new device is provided comprising:
at least one processor configured to execute the method steps according to any one of the above mentioned methods and/or method steps;
at least one display-device, configured to display the visual tasks and/or stimulating images to the user.

According to some embodiments, the device further comprising at least one input-device, configured to collect and interpret, at any given time-step (N=K), the user's response to the displayed images.

According to some embodiments, the device further comprising an apparatus selected from: a computer, a smart phone, a tablet and any combination thereof.

According to some embodiments, the device further comprising at least one of: data storage for the user's inputs and provided analyses, an input device, a speaker device, a microphone device, a computer mouse.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A, 1B, and 1C schematically illustrate examples of visual training-tasks, according to some embodiments of the invention;

FIGS. 2A, 2B, 2C, 2D, 2E, 2F, and 2G, schematically illustrate more examples of visual training-tasks, according to some embodiments of the invention;

FIG. 3 schematically illustrates devices and some of the method steps for presenting visual training-tasks, according to some embodiments of the invention;

FIG. 4 schematically illustrates devices and some of the method steps for presenting visual training-tasks, according to some embodiments of the invention;

FIGS. 5A, and 5B schematically illustrate examples of visual stimulations, for training or testing, according to some embodiments of the invention;

FIGS. 6A, 6B and 6C schematically illustrate examples of visual a visual training-task and their EEG readings;

FIGS. 7A, 7B, 7C and 7D schematically illustrate examples of EEG readings, after providing a user with training stimulating images of FIGS. 5A and 5B;

FIGS. 8A, 8B, 8C and 8D schematically illustrate examples of four visual training-stimulation and their EEG frequency readings;

FIGS. 9A, 9B, 9C and 9D schematically illustrate an example of a visual training-task (FIG. 9D) and its EEG readings, before and after training;

FIGS. 10A, 10B, 10C and 10D schematically illustrate an example of a visual test-stimulation (FIG. 10D) and its EEG frequency readings, before and after sessions of providing the visual training-tasks of FIG. 9D;

FIG. 11 schematically illustrate an EEG sensors' mapping;

FIG. 12 schematically illustrate an example of a visual task comprising working memory (WM) task, according to some embodiments of the invention;

FIGS. 13A and 13B demonstrate the results (accuracy and response time) of 12 participants, which were presented with tasks having one, two or three items (shapes);

FIGS. 14A and 14B demonstrate the Gamma coherence, for the same tasks and participants as in the results of FIGS. 13A and 13B;

FIGS. 15A, 15B, 15C and 15D demonstrate Gamma coherence before and after a training of two subjects;

FIGS. 15E, 15F, 15G and 15H, demonstrate resulted improvement in cognitive abilities, for the subjects as in FIGS. 15A-15D;

FIGS. 16A, 16B, 16C and 16D demonstrate P300 reaction difference (target vs. non-target), P300 magnitude and P300 latency, during a WM task, shown pre- and post-training of a subject;

FIGS. 16E and 16F demonstrate the relation between frequency power responses, in both P7+P8 and Fz, and blurriness presentations;

FIG. 17 demonstrates a “count the dogs” task, according to some embodiments of the invention; and

FIGS. 18A and 18B demonstrate the P300 response measured during the “count the dogs” task, as in FIG. 17.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

PCT application PCT/IL22/51147 published as WO2023/073715A2 is incorporated by reference herein, in its entirety.

According to some embodiments, cognitive decline is associated with impaired brain oscillations. Brain oscillations underlie the function of our brains, dictating how we both think and respond to the world around us. The synchronous activity of neurons generates these rhythms, which allow different parts of the brain to communicate and orchestrate reactions to internal and external stimuli. Over the past decades, extensive research has been initiated to examine changes in brain oscillations (rhythms) in age-related cognitive decline and Alzheimer disease (AD). Current evidence commonly supports the efficacy of resting-state EEG/MEG (rsEEG/MEG) as a non-invasive predicting biomarker of neuro-degeneration and conversion from a mild cognitive impairment (MCI) to AD.

According to some embodiments, ElectroEncephaloGraphy (EEG) is a method to record an electrogram of the electrical activity on the scalp that has been shown to represent the macroscopic activity of the surface layer of the brain underneath. It is typically non-invasive, with the electrodes placed along the scalp. EEG measures voltage (V) fluctuations resulting from ionic current (I) within the neurons of the brain. Clinically, EEG refers to the recording of the brain's spontaneous electrical activity over a period of time, as recorded from multiple electrodes placed on the scalp.

According to some embodiments, and as demonstrated in the following, EEG readings were recorded during tasks and/or image displays, using 30 dry (no gel added) channels, wireless headset by Cognionics® running at a sample rate of 500 Hz. Time Frequency Power analyses were performed in specific time windows (~0.5 sec) for gamma 30-50 Hz band frequency.

According to some embodiments, diagnostic applications generally focus either on event-related potentials, or on the spectral content of EEG. The former investigates potential fluctuations time locked to an event, such as ‘stimulus onset’ or ‘button press’. The latter analyses the type of neural oscillations (popularly called “brain waves”) that can be observed in EEG signals in the frequency domain.

According to some embodiments, the brain's electrical charge is maintained by billions of neurons. Scalp EEG activity shows oscillations at a variety of frequencies. Several of these oscillations have characteristic frequency ranges, spatial distributions and are associated with different states of brain functioning. These oscillations represent synchronized activity over a network of neurons.

According to some embodiments, P300 is a biomarker for cognitive function, reflecting the processing speed of neurons in reaction to a basic stimulation requiring a cognitive decision.

According to some embodiments, P300 is measured using EEG as the positive brainwave component in brain activity measured about 300 milliseconds (mSec) after stimulus onset (P300a), differentiating target and non-target stimulation affected mainly by attention, or by slower cognitive processing (about 400-500 mSec) such as short-term memory or decisions (P300b). P300 might generate from hippocampus, amygdala, thalamus and basal ganglia. It could be used as a measuring tool of cognitive dysfunction in many neurological disorders, and it is especially related to attention and short-term memory. It reflects an early cognitive change in MCI and predicts dementia in elderly Alzheimer's disease.

According to some embodiments, one biomarker is the power of the Gamma oscillatory activity (20-80 Hz). According to some embodiments, to calculate the gamma power, one should compare one measure (e.g., memory task) to baseline. Thus, it is 10 log(memory case/baseline). According to some embodiments, the gamma oscillatory activity contributes to a wide range of human cognitive functions, such as attention, perception, object recognition, memory processes, face recognition, and emotional paradigms, from which it can be inferred that gamma synchronization is a fundamental process for many brain functions. Reduction in gamma power is demonstrated with age, in normal healthy aging, in occipital parietal, in frontal areas. Reduced resting-state Gamma power/synchronization and delayed Gamma reaction, and increased gamma-band power or connectivity (i.e., cross-frequency coupling (CFC)) are demonstrated in MCI and AD groups compared to normal healthy aging.

According to some embodiments of the invention, a new visual task and/or stimulation method is provided configured for inducing persistent and stimulation-independent changes in subject's brain neural oscillations. The currently provided method is a training method via a novel visual-task and/or -stimulation, configured to induce and/or evoke long-lasting changes in brain oscillations that are not restricted to the stimulation type. These changes modulate neuronal activity leading to persisting changes that generalize to untrained functions and stimulations.

Particularly, the new training method is configured for an increase in the power of the gamma oscillation, in reaction to the provided visual task and/or stimulations. Following the completion of the training, users have demonstrated an increase in the power of the gamma oscillation, versus the baseline for such pre-training parameters. The training also results in an increase in the gamma power oscillation, in reaction to different types of visual task and/or stimulations, that were not included in the training process (Untrained Stimuli).

According to some embodiments, the currently provides visual-task and/or -stimulation training method is configured to induce changes in brain waveform that, result in improvement of P300 (increasing amplitude and/or shortening the latency), compared with its pre-training baseline, wherein this change/improvement is not restricted to specifically training provided visual task and/or stimulations.

According to some embodiments, Amplitude (μVolt) is defined as the difference between the mean pre-stimulus baseline voltage and the largest positive-going peak of the Event-Related potential (ERP) waveform within a time window (e.g., 250-500 mSec, although the range can vary depending on stimulus modality, task conditions, subject age, etc.). Latency (mSec) is usually defined as the time from stimulus onset to the point of maximum positive amplitude within a time window. The neuropsychological origins of the P300a and P300b subcomponents are as follow: The neural loci of P300a and P300b generation are different, and a cognitive model notes that P300a originates from stimulus-driven frontal attention mechanisms during task processing, whereas P300b originates from temporal-parietal activity associated with attention and appears related to subsequent memory processing.

According to some embodiments, the phrases “brain wave” and “neural oscillation” as used herein refer to electrical-potentials or electrical-impulses given off by brain tissue. According to some embodiments, the phrase “frequency power” as used herein refers to the electric power of measured neural oscillation. According to some embodiments, the measure of frequency power is provided by convolution (Fast Fourier Transform) of the waveform, and usually is presented in units of decibels [dB].

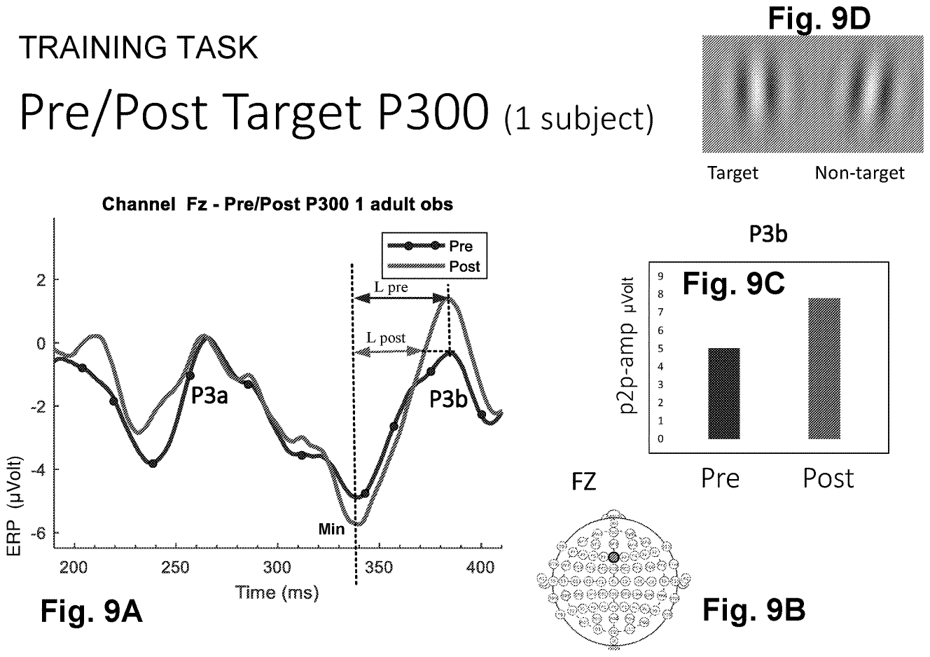

According to some embodiments of the invention, the phrase “amplitude” as used herein refers to the difference between the P300b maximum value and the preceding minimum value, as demonstrated in FIGS. 9A and 9C.

According to some embodiments of the invention, the phrase “latency” as used herein refers more specifically to “P300b-latency”, which is the time difference between the P300b maximum and the preceding minimum, as demonstrated in FIG. 9A.

According to some embodiments of the invention, and as used herein, the phrase “response” refers to the user's active reply for the requested task.

According to some embodiments of the invention, and as used herein, the phrase “reaction” refers to the user's brain-wave readings and/or brain induced activity, in reaction to the requested task and/or the presented stimulation.

According to some embodiments of the invention, a new method is provided for increasing and/or improving a user's cognitive abilities, the method comprising presenting to the user one or more visual training-tasks, via processor implemented method steps of displaying, at least one session (S₁, S₂ . . . S_{M}), each session of K_{S} visual training-tasks, requiring the user's one or more responses; wherein the visual training-tasks are configured to induce an amplification of the brain's frequency power, in reaction to at least the provided visual training-tasks.

According to some embodiments of the invention, a new method is provided for increasing and/or improving a user's cognitive abilities, the method comprising presenting to the user one or more training stimulating-images, via processor implemented method steps of displaying, at least one session (S₁, S₂ . . . S_{M}) each of K_{S} training stimulating-images; wherein the training stimulating-images are configured to induce an amplification of the brain's frequency power, in reaction to at least to the provided training stimulating-images.

According to some embodiments of the invention, a new method is provided for increasing a user's brain coherence between visual and cognitive regions, the method comprising presenting one or more visual training-tasks via processor implemented method steps of displaying, at least one session (S₁, S₂ . . . S_{M}) each of K_{S} visual training-tasks to the user; wherein the visual training-tasks are configured to induce an increase in the power of the brain's Gamma wave at local regions, in reaction to at least the provided visual training-tasks.

According to some embodiments of the invention, a new method is provided for increasing a user's brain coherence between visual and cognitive regions, the method comprising presenting one or more training stimulating-images via processor implemented method steps of displaying, at least one session (S₁, S₂ . . . S_{M}) each of K_{S} visual-stimulations to the user; wherein the training stimulation-images are configured to induce an increase in the power the brain's Gamma wave at local regions, in reaction to at least the provided training stimulating-images.

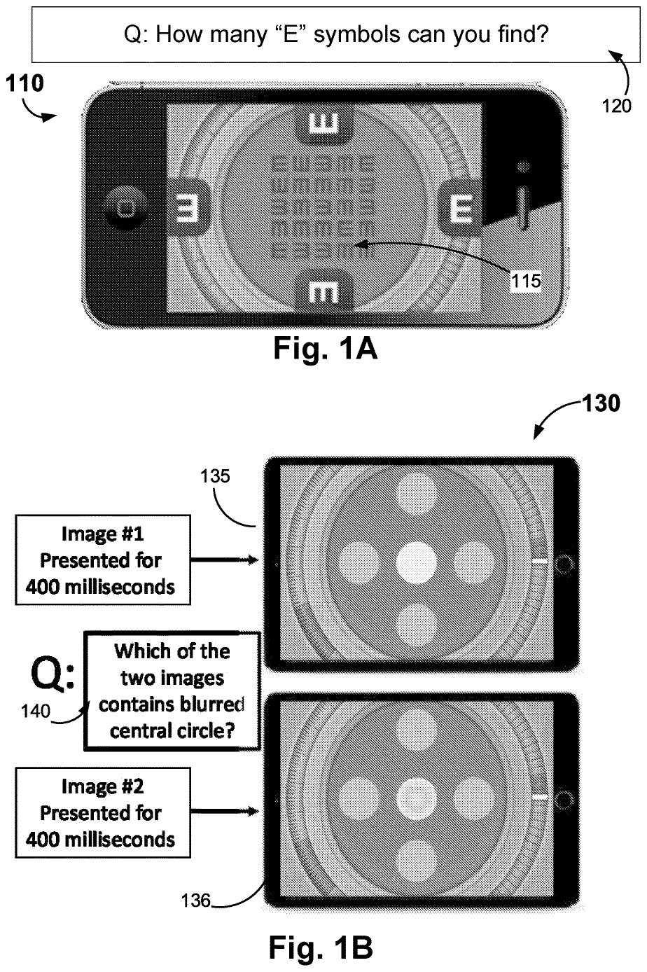

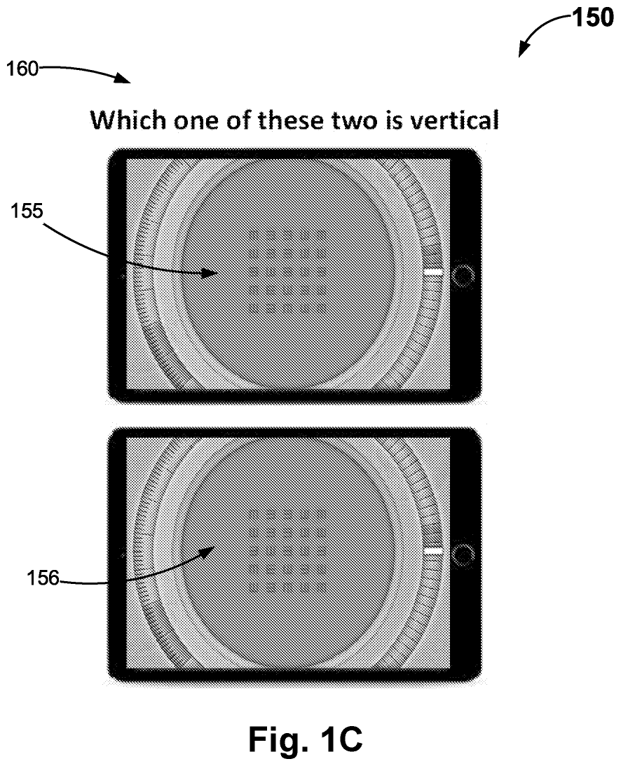

According to some embodiments of the invention, and as demonstrated for example in FIGS. 1A-1C, 2A-2G, 3 and 4, a new method [300,400] is provided for presenting one or more visual tasks to a user. The method comprising processor implemented method steps of displaying [310,410], at least one session of K_{S} visual tasks to the user, requiring the user's one or more responses.

According to some embodiments, the visual tasks are selected such that the requested responses are configured to provide a long-term improvement of one's response, by means of better cognitive- and/or motoric-responses, at least to the provided visual task, and optionally to other visual tasks as well.

According to some embodiments of the invention, and as demonstrated for example in FIGS. 1A-1C, 2A-2G, 3 and 4, a new method [300,400] is provided for presenting one or more visual tasks to a user. The method comprising processor implemented method steps of displaying [310,410], at least one session of K_{S} visual tasks to the user, requiring the user's one or more responses; wherein the visual tasks are selected such that the requested responses are configured to stimulate and/or induce a long-term amplification of the brain's frequency power, in reaction to at least to the provided visual tasks, and optionally to other visual tasks as well.

According to some embodiments of the invention, the above-mentioned session/s of visual tasks are configured for training a user, to improve one's responses and/or brain reaction.

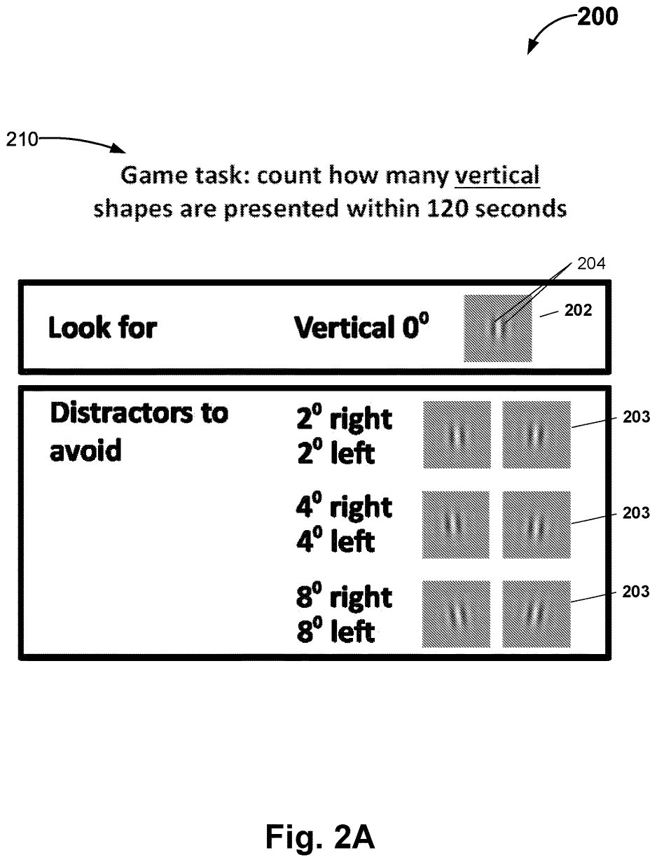

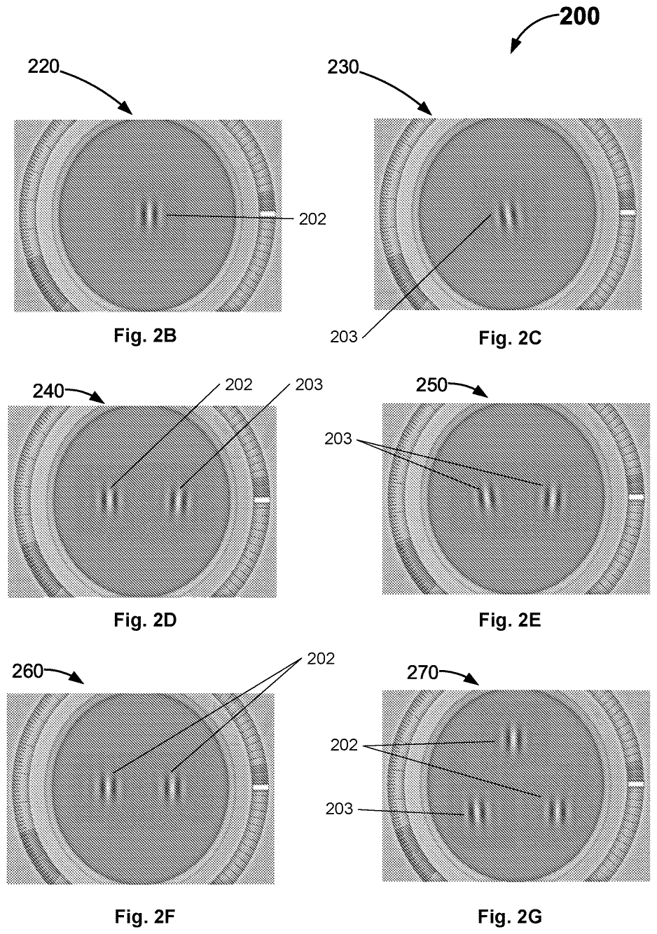

According to some embodiments, the visual task [110] comprises at least one image [115], as demonstrated in FIG. 1A. According to some embodiments, the visual task [130] comprises at least two images [135 & 136, 155 & 156], as demonstrated in FIGS. 1B and 1C. According to some embodiments, the visual task [200] comprises a plurality of images and/or tasks [220-270], as demonstrated, for example in FIGS. 2A-2G.

According to some embodiments, the method comprises providing instructions to the user, in regard to the provided visual task/s. According to some embodiments, the instruction is general and same for all sessions and all time-steps. According to some embodiments, the instruction can be changed per each session. According to some embodiments, the instruction can be changed per each time-step. According to some embodiments the instruction may be provided to the user verbally, by a monitoring personal. According to some embodiments the instruction may be provided to the user via a processor and at least one device selected from: a display device, a speaker.

According to some embodiments, the visual task/s comprises at least one instruction to the user that requires one's response. As demonstrated, for example, in FIG. 1A [120], FIG. 1B [140], FIG. 1C [160], and FIG. 2A [210].

According to some embodiments, the user's response comprises at least one of the following:
a cognitive response; non-limiting examples include: an answer for a question (e.g., yes/no, true/false, number of counted target-items), item selection by criteria, and more;
a motoric response; non-limiting examples include: eye/limb motion (e.g., follow an item with a curser, catch an item with the curser);
an emotional response (e.g., what do you feel?);
a behavioral response (e.g., what would you do?);
a reflex response (e.g., follow an item with a curser, catch an item with the curser), which may involve both cognitive- and motoric-response; and
eye response (e.g., eye movement, eye blink, pupil dynamics).

According to some embodiments, the phrase “long-term” refers to brain wave changes that persist more than an hour, and up to few months.

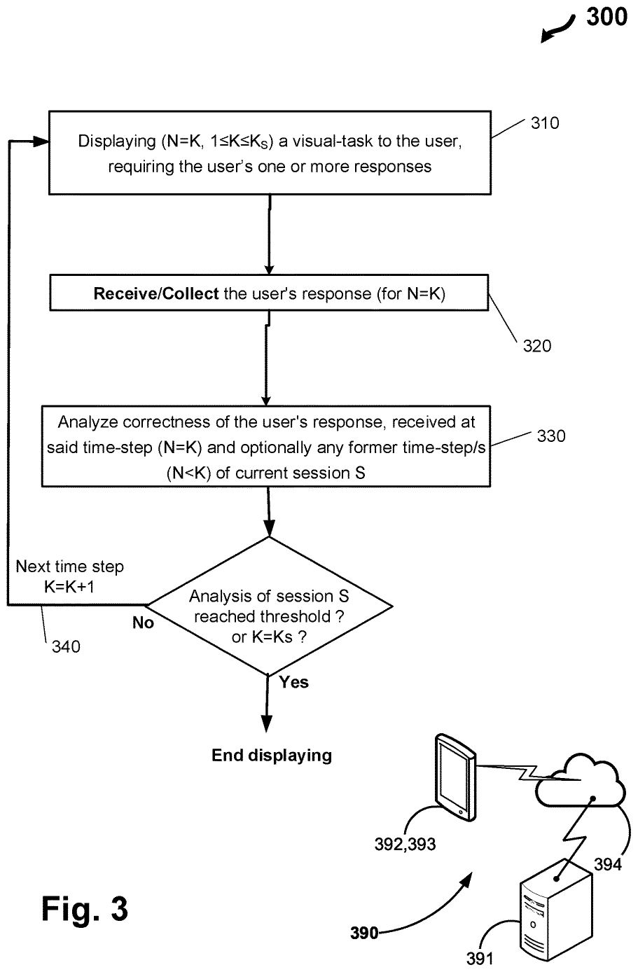

According to some embodiments, and as demonstrated for example in FIG. 3, the method/s [300] further comprising:
receiving and/or collecting [320], at a given time-step N (N=K, 1≤K≤K_{S}) of a session S₁ having K_{S} time steps, the user's response-profile to a displayed visual task, displayed at the given time step (N=K);
analyzing [330] the user's response-profile, received at the given time-step (N=K) and optionally any former time-step/s (N<K) of the session S₁;
repeating [340] the step of displaying, at following time-step/s (K=K+1) of the session S₁, a predetermined number of times K≤K_{S} for the session S₁ and/or until the response-profile has reached a predetermined threshold.

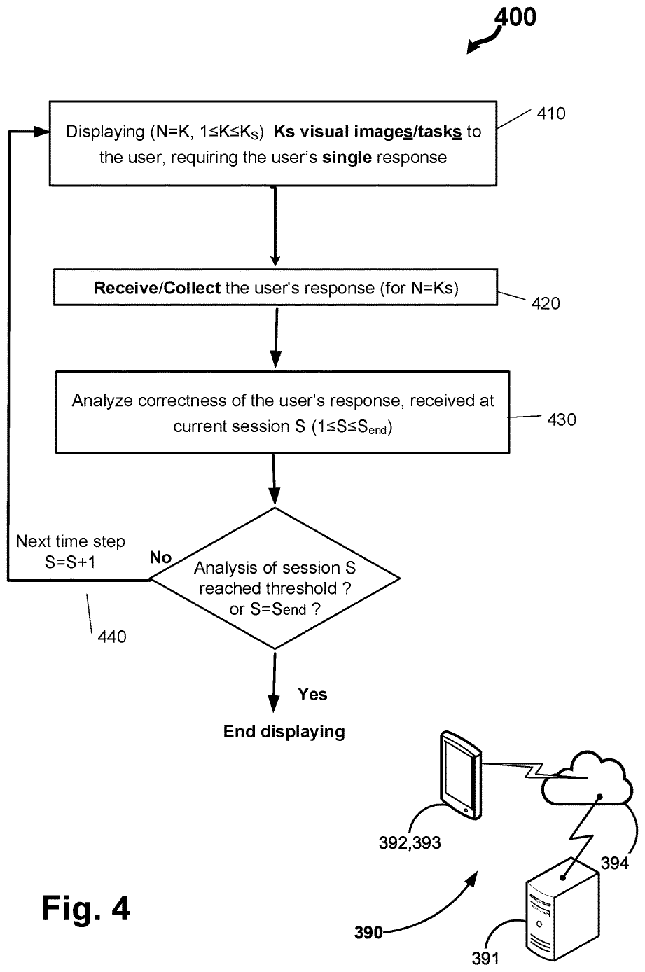

According to some embodiments, and as demonstrated for example in FIG. 4, the method/s [400] further comprising:
receiving and/or collecting [420], at a last time-step (N=K_{S}) of a session S₁ having time-steps (N=K, 1≤K≤K_{S}), the user's response-profile to a plurality of displayed visual tasks, displayed at time steps N (N=K, 1≤K≤K_{S}) of session S₁;
analyzing [430] the user's response-profile, received at the last time-step (N=K_{S}) of session S₁;
repeating [440] the steps of displaying, at following time-steps N (N=K, 1≤K≤K_{S}) of a following session S₂, a predetermined number of sessions and/or until the response-profile has reached a predetermined threshold.

Reference is now made to FIGS. 2A-2G. FIG. 2A demonstrates the instruction to the user, to identify how many shapes have vertical lines [204], a correct shape is demonstrated with reference [202], tilted lines for an incorrect response is demonstrated with reference [203]. The tasks [220-270] of FIG. 2B-2G, can be used via both method algorithms [300] and [400]. For example, a response can be collected [320] and analyzed [330] after each task/image separately, according to method [300], having 6 responses to collect and analyze; or, collect and analyze a single response, after all tasks/images [220-270] were presented, were the user is requested to keep counting the shapes having vertical lines [202] from all images/tasks, according to method steps [420] and [430] of method [400].

According to some embodiments, the response-profile comprises at least one of the following:
correctness of an answer (e.g., correct/incorrect);
response quality (e.g., correct percentage); according to some embodiments, the percentage of the correct response is measured per each type of task;
response time, i.e., the time it takes to respond to each task; according to some embodiments, a time profile is analyzed per each type of task.

According to some embodiments, the method further comprises measuring the user's brain signals via an EEG device and analysis thereof. According to some embodiments, the EEG measuring and analysis are provided after the training. According to some embodiments, the EEG measuring and analysis are provided during the training method. According to some embodiments, the EEG measuring and analysis are provided before the training method. According to some embodiments, the EEG measuring and analysis are provided before and/or during and/or after the training method.

According to some embodiments, the measured EEG readings and their analysis are used for selecting the visual tasks and/or images for at least some of the disclosed methods, in accordance with the goal of its related training and/or treatment. In such embodiments, the visual tasks and/or images are selected via examining the brain's reaction to the presentation of the visual tasks and/or images, during the time of the presentation and/or after presentation and/or long-term after presentation. In some embodiments, via comparison with readings measured before the presentation of visual tasks and/or images.

According to some embodiments, the measured EEG readings and their analysis are provided per a specific user and are used for selecting the visual tasks and/or images, in accordance with the goal of its related training and/or treatment, for training and/or treating said specific user.

According to some embodiments, the measured EEG readings are provided from plurality of subjects and their analysis is provided for selecting the visual tasks and/or images, in accordance with the goal of its related training and/or treatment, for training and/or treating of any user.

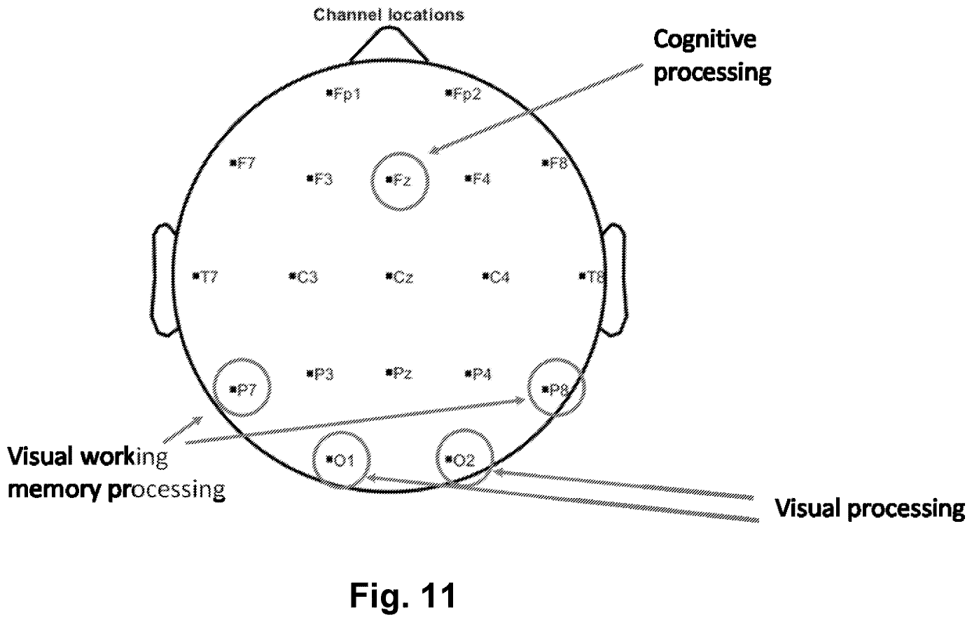

According to some embodiments, the analyzed EEG readings are provided via a sensor channel selected from: Fz, P7, P8, O1,O2 and any combination thereof (demonstrated in FIG. 11). According to some embodiments the analysis of the readings of channel Fz is configured to evaluate cognitive processing. According to some embodiments, the analysis of the readings of channels P7 and/or P8 is configured to evaluate visual working memory processing. According to some embodiments, the analysis of the readings of channels O1 and/or O2 is configured to evaluate visual processing.

According to some embodiments, the measured and/or analyzed EEG readings comprise frequency power. According to some embodiments, the induced long-term amplification of the brain's frequency power, in reaction to provided visual tasks (i.e., tasks provided at training sessions, or other tasks) and or/images, is measured and/or analyzed via the EEG readings. According to some embodiments, the selecting of visual task and/or images, for inducing long term amplification of the brain's frequency power, is based on the analysis of the measured EEG readings.

According to some embodiments, the measured and/or analyzed EEG readings comprise Gamma waves. According to some embodiments, the selecting of visual task and/or images for inducing long term increase of the power of the brain's Gamma wave (as a whole and/or at local regions of the brain), is based on the analysis of the measured EEG readings.

According to some embodiments, the measured and/or analyzed EEG readings comprise P300 positive brainwave component. According to some embodiments, the selecting of visual task and/or images, for amplifying of the P300 positive brainwave component, is based on the analysis of the measured EEG readings.

According to some embodiments, the measured and/or analyzed EEG readings comprise the P300 latency of the brain's reaction. According to some embodiments, the selecting of visual task and/or images, for shortening of the P300 latency of the brain's reaction, is based on the analysis of the measured EEG readings.

According to some embodiments, the measured and/or analyzed EEG readings comprise coherence, which refers to synchronization across brain regions. According to some embodiments, the coherence is measured between visual processing regions and cognitive processing regions, as the coherence level involves with cognitive processes that require long-distance brain network coordination; as demonstrated for the Gamma wave in FIG. 2 of Changes of Functional and Directed Resting-State Connectivity Are Associated with Neuronal Oscillations, ApoE Genotype and Amyloid Deposition in Mild Cognitive Impairment, Michels et. al., Frontiers in Aging Neuroscience (2017).

According to some embodiments, EEG coherence between two electrodes measures the similarity or synchronization of electrical activity, at those specific locations in the brain. To ensure accurate coherence estimation of EEG, some data preprocessing steps can be included.

A non-limiting example for coherence calculation includes: first, removing noise and artifacts, including eye blinks using a cutoff filter a 180 μVolt threshold on the mean global field potential and an additional 70 μVolt on the mean of the frontal electrodes. Then the data was filtered to isolate the desired frequency band of interest (e.g., gamma 30-50 Hz). Then computing the cross-spectral density between the signal in of the two distant scalp sites (i.e., Fz electrode for the frontal site and electrodes P7 and P8 for the parietal site on both sides of the cortex). This accounts for the phase relationship between the two signals. Finally, the coherence value is calculated by dividing the magnitude of the cross-spectral density by the square root of the product of the power spectra of the two signals. This normalization accounts for differences in the overall power levels between the two signals. Coherence between two waveforms x and y was calculated spectrally as:

$${γ}_{xy}^{2} ( f ) = {[ {G}_{xy} ( f ) ]}^{2} / [ {G}_{xx} ( f ) {G}_{yy} ( f ) ] ,$$

where G_{xy}(f) is the mean cross-power density and Gₓₓ(f) and G_{yy}(f) are the respective mean auto-power spectral densities (Jiang, Z. yan. Study on EEG power and coherence in patients with mild cognitive impairment during working memory task. J. Zhejiang Univ. Sci. B. 6, 1213-1219; 2005).

According to some embodiments, the above-mentioned response-profile comprises the EEG readings and/or their analysis, according to any one of the above mentioned methods.

According to some embodiments, the predetermined threshold is at least one selected from:
a predetermined number of sessions;
a predetermined level of response-correctness, -quality and/or -time; and
saturation of the measured level of brain reaction.

According to some embodiments, the visual task includes at least one of: attention request, perception task, object recognition task, memory task, face recognition task, and emotional incentive task.

According to some embodiments, the visual task comprises differentiating between displays of target-image/s [136] and non-target image/s [135], as demonstrated for example in FIG. 1B.

According to some embodiments, the inducement of the amplification of the brain's frequency power, comprises at least one of:
amplification of an electric peak (e.g., P300b, marked as P3b, FIG. 9A,9C); and
advancing (shortening) of the latency (L) of the brain's reaction, after (post) the provided visual task (e.g., P300b, marked as P3b, FIG. 9A, Lₚₒₛₜ<Lₚᵣₑ).

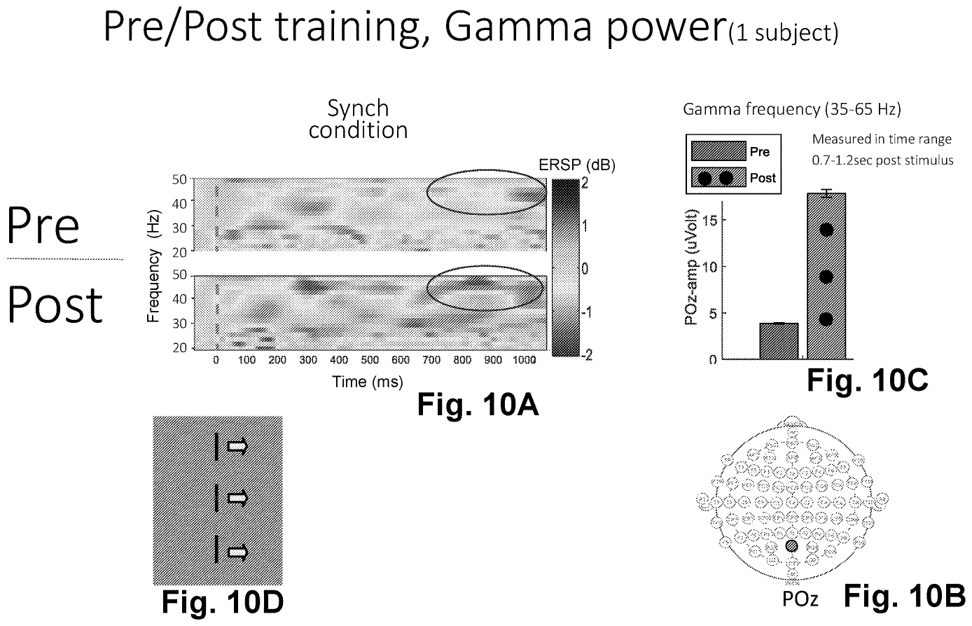

According to some embodiments, the training visual task/s (e.g., FIG. 9D to recognize the target image) are selected and configured to induce a long-term amplification of the brain's frequency power in reaction at least one new visual task (e.g., passive task as in FIGS. 10D, presenting synchronized motion of images), which was not provided to the user at the training sessions; accordingly the training visual tasks (FIG. 9D) are configured to induce a long-term amplification to the brain's frequency power in reaction to a new test visual tasks (FIG. 10D) and/or a new test visual-stimulations.

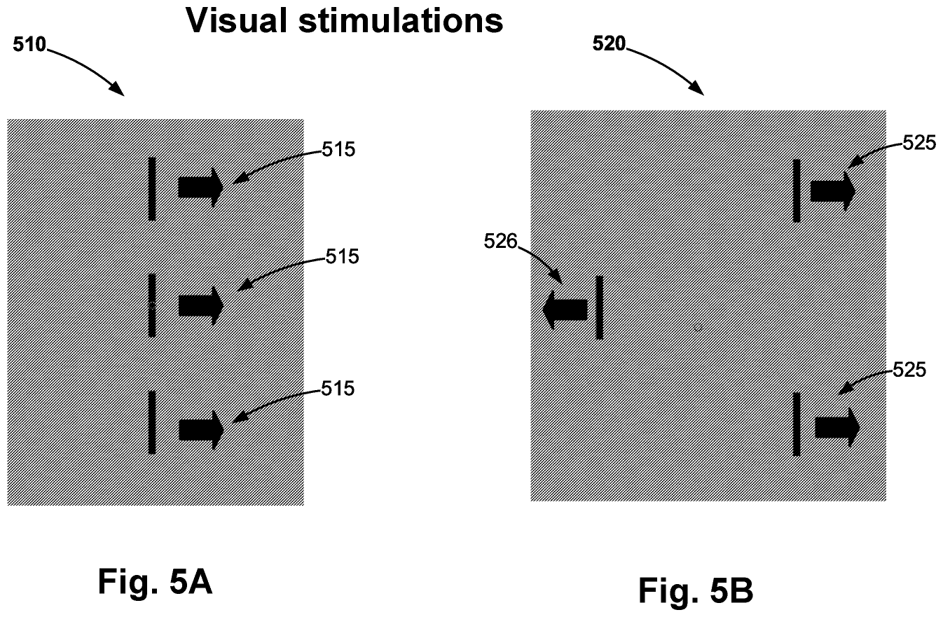

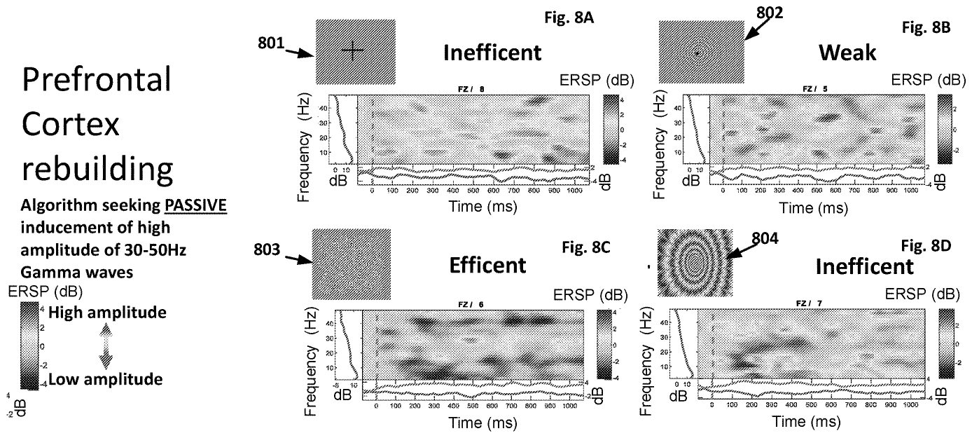

According to some embodiments of the invention, a new method is provided for presenting one or more visual stimulations (e.g., stimulating image) to a user; passive stimulations, with no requested task to be performed by the user. The method comprising processor implemented method steps of displaying, at least one session of K_{S} stimulations to the user. The visual-stimulations are selected to stimulate and/or induce a long-term amplification of the brain's frequency power in reaction to at least the provided visual-stimulations, and optionally to other visual-stimulations as well. Examples are demonstrated in FIGS. 5A, 5B, 8A (801), 8B (802), 8C (803), and 8D (804).

According to some embodiments, the visual stimulation (e.g., stimulating image) comprises an image with at least one dynamic element; dynamic by means of moving elements during a presentation. FIGS. 5A and 5B demonstrate a visual stimulation with several dynamic images (shown here with arrows for indicating the elements moving-direction). FIG. 5A demonstrates a synchronized stimulation [510], according to some embodiments, where all elements [515] are moving in the same direction and same velocity. FIG. 5B demonstrates an a-synchronized stimulation [520], according to other embodiments, where at least one of the elements [526] is moving in a different direction and/or a different velocity than at least one other element [525].

According to some embodiments, the induced long-term amplification of the brain's frequency power, in reaction to the provided visual tasks and/or stimulations, is configured for improving a condition of a patient suffering from at least one of: poor cognitive response, poor motoric response, Alzheimer, depression, schizophrenia, ADHD, dyslexia, Parkinson, multiple sclerosis and any combination thereof.

According to some embodiments, a device [390] is provided configured to present images to a user, comprising:
at least one processor [391] configured to execute the method steps according to according to any one of the above mentioned method steps;
at least one display-device [392], configured to display the visual tasks and/or stimulations to the user, according to one any of the above-mentioned embodiments.

According to some embodiments, the device further comprises at least one input-device [393], configured to collect and interpret, at any given time-step (N=K), the user's response to the displayed images.

According to some embodiments, the device comprises an apparatus selected from: a computer, a smart phone, a tablet and any combination thereof.

According to some embodiments, the device further comprises at least one of: data storage [394] for the user's inputs and provided analyses, an input device, a speaker device, a microphone device, a computer mouse.

### Experiments and Results

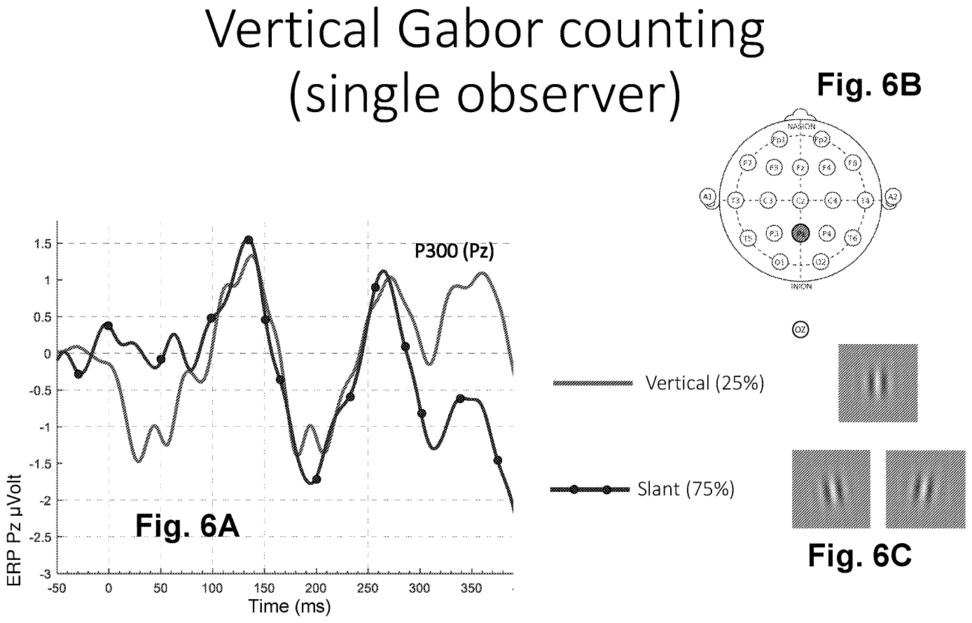

Reference is now made to FIGS. 6A-10D, demonstrating EEG results and analysis, for at least some of the above-mentioned methods.

FIG. 6A demonstrates EEG P300 reading components (Voltage vs. time), from EEG sensor P_{Z} (FIG. 6B), related to pre-frontal cortex. The readings are measured during a task such as in FIG. 6C, requesting a user to identify the shapes having vertical images (the upper image, not the lower images). The graph-lines in FIG. 6A demonstrate the brain reaction; when displaying to the user vertical lines (0°), in this test it was 25% of presentations, and the reaction is marked with a simple graph-line; when displaying to the user tilted lines the reaction, in this test it was 75% of presentations, the reaction is marked with dotted graph-line. According to the demonstrated examples, one can see that, easier decisions, such as no-tilt (0°) are more stimulating to the brain than difficult decisions (tilted). Accordingly, the brain waves for the non-tilted image (“target”) are remarkably different than the tilted images and can serve as a measure (marker) for cognitive abilities of deployment of attention specifically to the task.

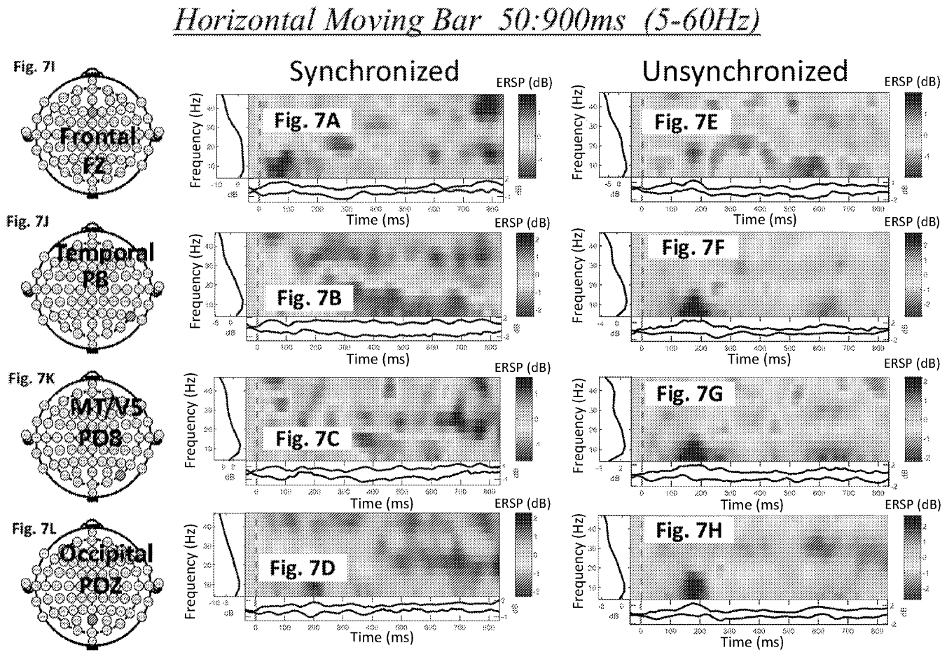

FIG. 7A-7H demonstrate EEG analyzed Frequency vs. Time vs. colored Amplitude (low=GREEN, high=RED), after the visual stimulation [510,520] display to a user as in FIGS. 5A and 5B, where each panel shows results from a different electrode: FIGS. 7A and 7E demonstrate measurements via the EEG frontal FZ sensor, shown in FIG. 71; FIGS. 7B and 7F demonstrate measurements via the EEG temporal P8 sensor, shown in FIG. 7J; FIGS. 7C and 7G demonstrate measurements via the EEG MT/V5 P08 sensor, shown in FIG. 7K; and FIGS. 7D and 7H demonstrate measurements via the EEG occipital POZ sensor, shown in FIG. 7L; and where:
FIGS. 7A-7D: demonstrate the user's brain reaction to the presented stimulation of shapes with vertical elements [515] moving in synchrony [510](left or right);
FIGS. 7E-7H: demonstrate the user's brain reaction to the presented stimulation of shapes with vertical elements [515,516] moving in a-synchrony [520](left or right);

Accordingly, one can see that movements in synchrony [510](FIGS. 7A-7D) show more gamma band (more red/dark areas) than the a-synchrony movements [520](FIGS. 7E-7H) which are less stimulating to the brain than the synchronized movements.

FIGS. 8A-8D demonstrate EEG analyzed P300 readings of: Frequency vs. Time vs. colored Amplitude (low=GREEN, high=RED), during and after four visual stimulations of passive-image displays, respectively 801-804, (no tasks) to a user. FIG. 8C, demonstrates that the stimuli image [803] evokes larger gamma frequency than the other images, [801,802,804]. Image [803] evokes gamma frequency in a range of 40 Hz and above (Y axis), accordingly inducing higher activity (namely, more areas that are colored red).

FIGS. 9A-9D disclose and demonstrate a training visual task and its EEG readings. A healthy 68 year old user (with no cognitive impairment) was trained via 90 training sessions (each session of 15 minutes, per day). The user was requested to distinguish between shapes having vertical images and tilted images, as in FIG. 9D. FIG. 9A demonstrates the pre-training (dotted graph-line) and post-training (simple graph-line) P300 readings measured via the EEG frontal Fz sensor (shown in FIG. 9B). FIG. 9C demonstrates the amplitude increase between the P3b peak and its preceding minimum value, pre-training vs. post training. The latency shortening is demonstrated in FIG. 9A where Lₚₒₛₜ<Lₚᵣₑ, showing that the amplitude of the post P300a reached to the same peak level of the pre P300a earlier (by about 15 mSec), and continue to increase the amplitude.

FIGS. 10A-10D disclose an example of a visual test-stimulation (FIG. 10D) and its EEG frequency readings, before and after sessions of providing the visual training-tasks of FIG. 9D, for the same healthy 68 year old user. The new unseen visual test-stimulation (as in FIG. 10D) was provided to the user after the 90 training sessions (each session of 15 minutes, per day). FIG. 10A demonstrates pre-training (upper panel) and post-training (lower panel) frequency measurements, measured via the EEG PO_{Z} sensor (shown in FIG. 10B). FIG. 10C demonstrates the Gamma frequency integral, at the time slot of 0.7 mSec and 1.2 mSec (circled; to show the time range of the analysis) and its increase between pre-training vs. post training. The gamma band at this time window increased after training as showing in the bar chart (FIG. 10c).

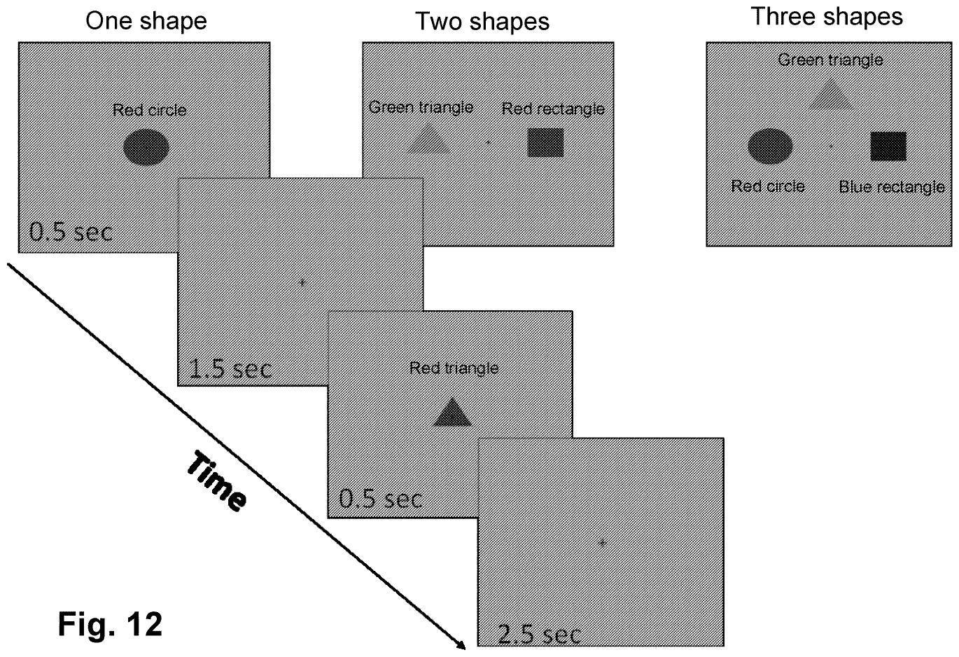

FIG. 12 discloses an example of a visual task comprising working memory (WM) task, according to some embodiments of the disclosure. In this task one, two or three colored items (e.g., shapes) were presented in one figure, at each timestep; (4 optional shapes×6 optional colors). FIG. 12 further presents an example for a task's timeline having, in this example, only one shape for a set of two presentations (each with a single item). In this example, the user is requested to remember and report if a specific shape in a specific color (“target”) was displayed; for example, the red triangle, after a set of two displays, in this example. In the demonstrated time-line:
a red circle (non-target) is presented for 0.5 Sec, and then disappears;
a cross stays on for 1.5 Sec, as a pause;
then a red triangle (target) is presented for 0.5 Sec and then disappears;
a cross stays on for 2.5 Sec as a second pause.
   the question to the user/subject is only whether the target appeared or not.

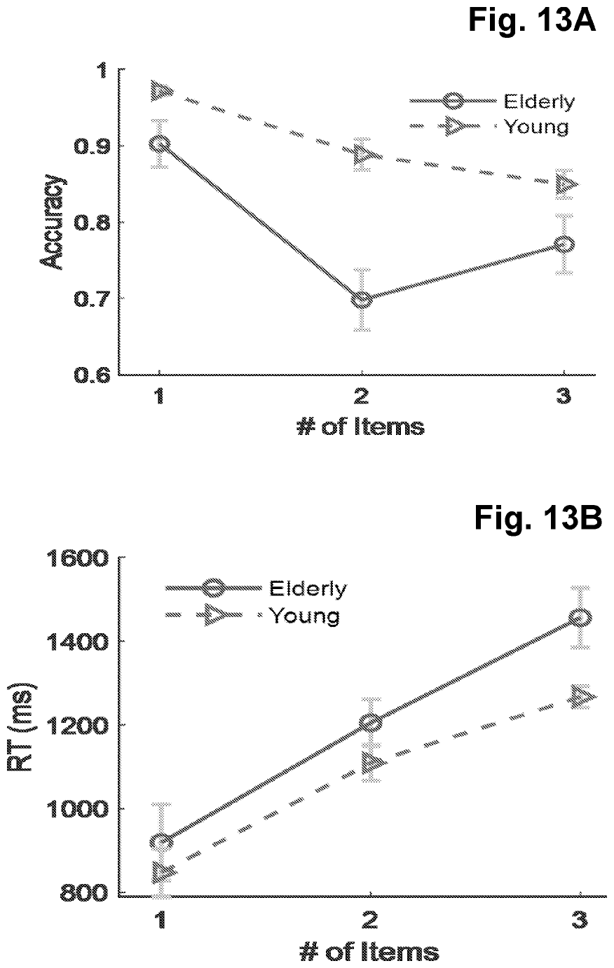

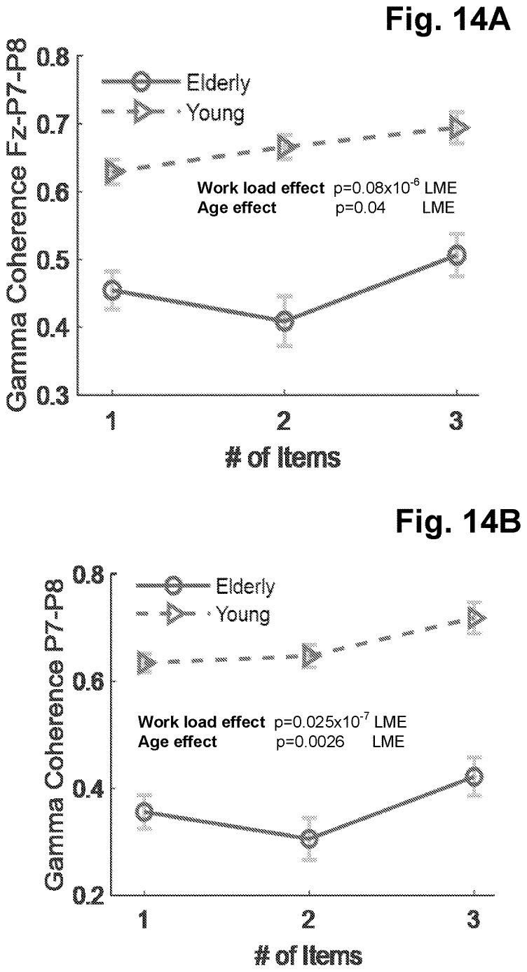

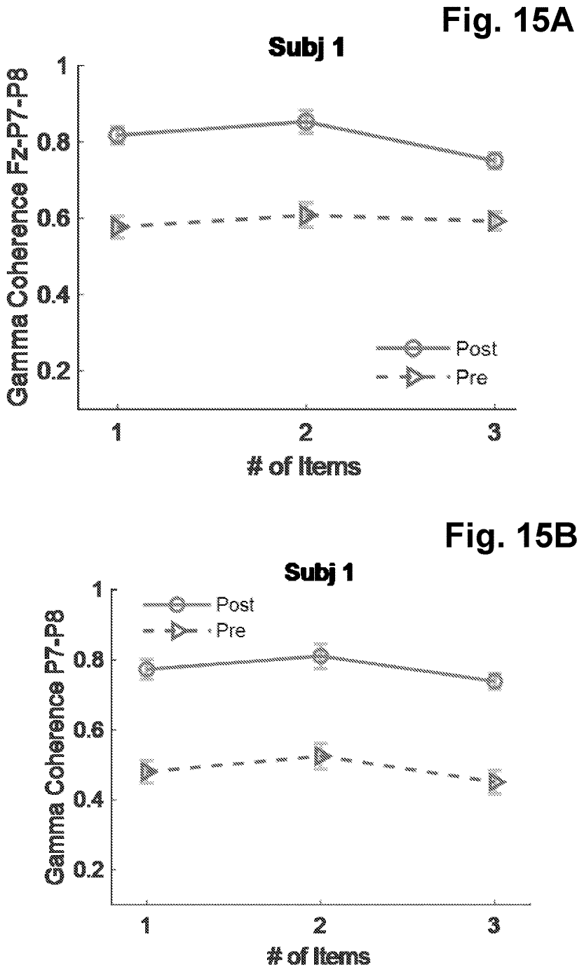

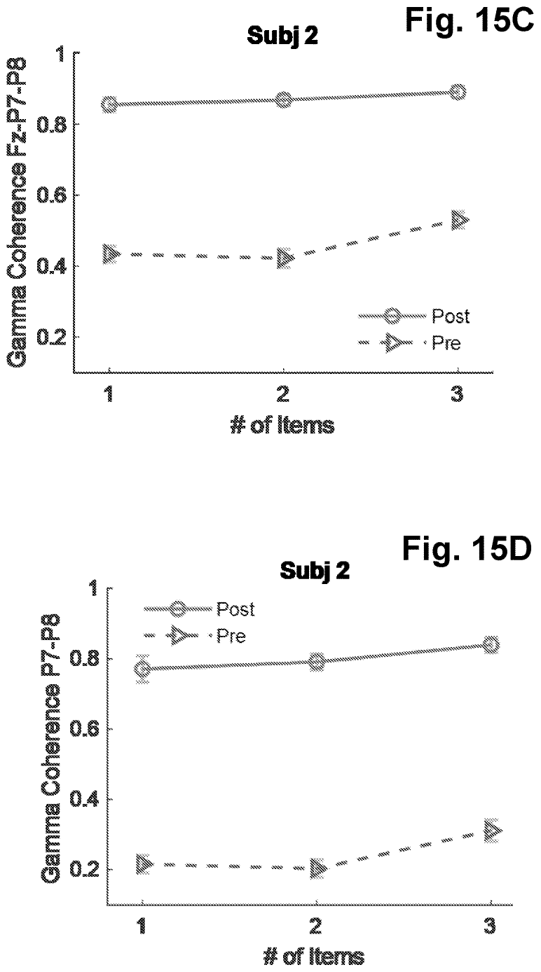

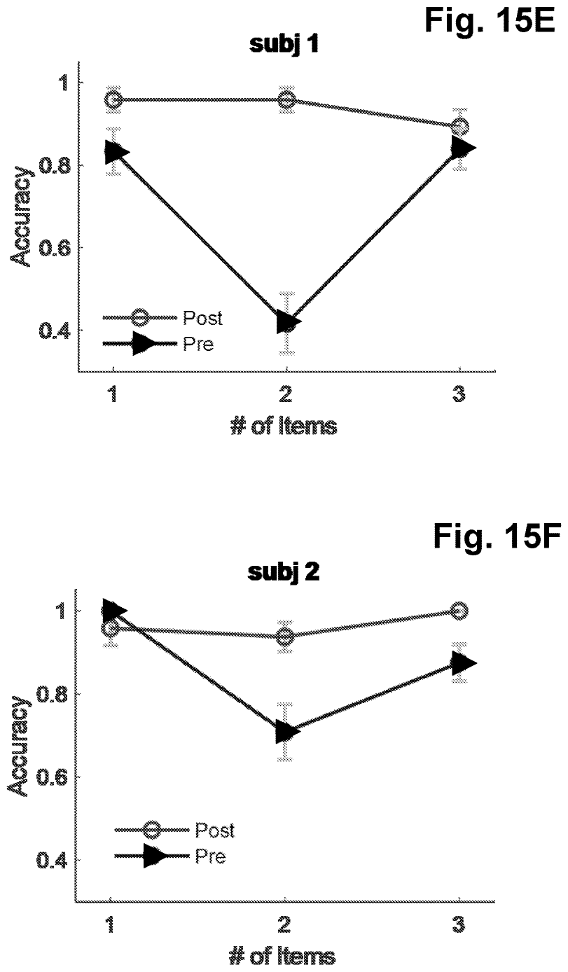

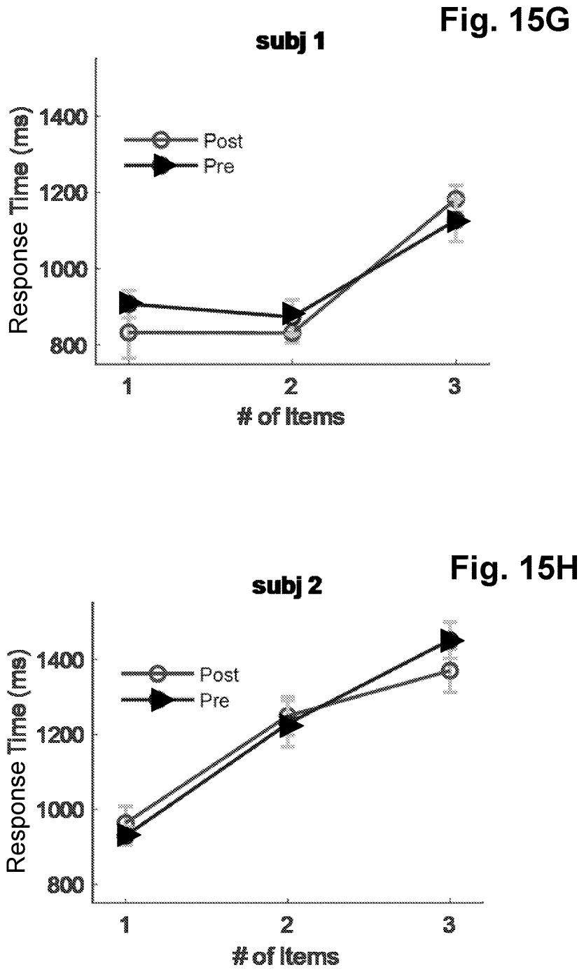

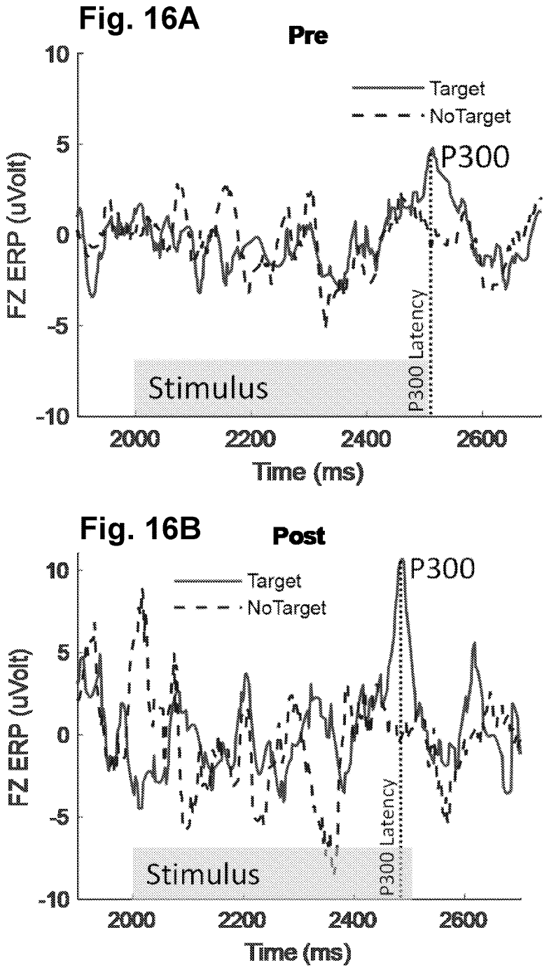

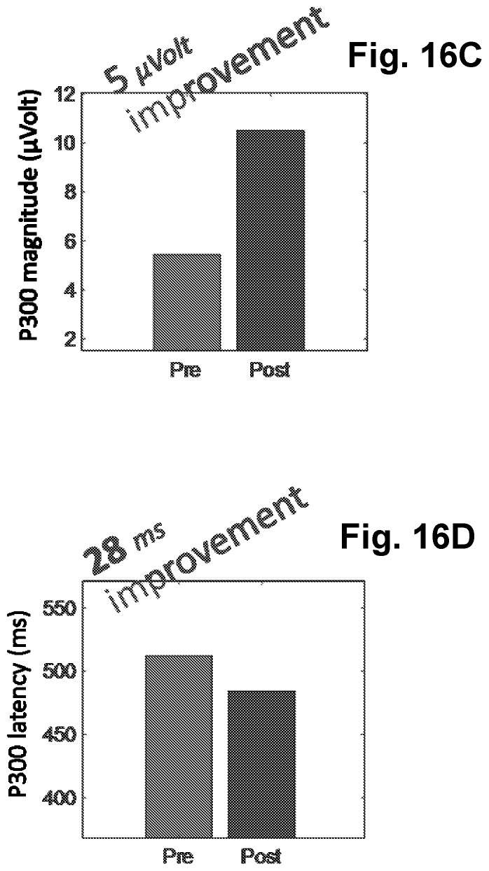

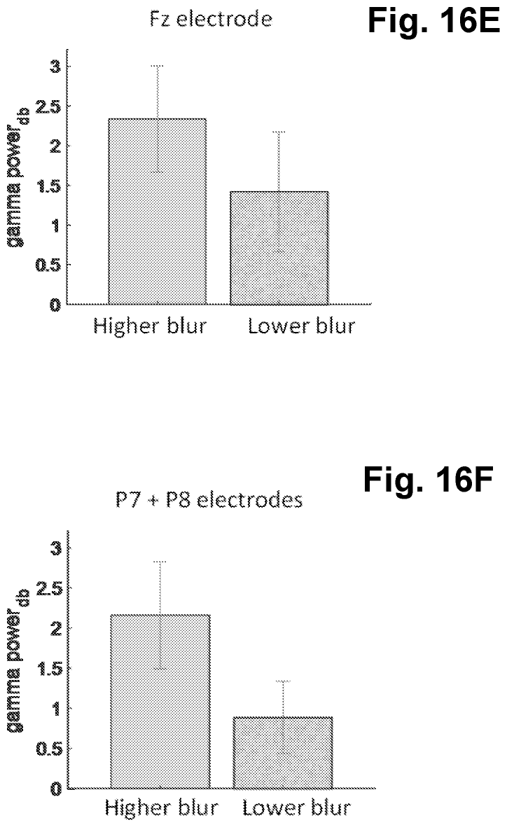

Similar experiments were conducted with participants (n=12, 6 young <40 and 6 old >60) who were instructed to memorize the shape and the color of the target image; and then report whether they have noticed a match. The test (no target) and the target were presented for 0.5 sec. The test's color and/or the shape were changed randomly. EEG readings were recorded during a shape-color-matching task, using 30 dry (no gel added) channels, wireless headset by Cognionics® running at a sample rate of 500 Hz. Time Frequency Power analyses were performed in specific time windows (~0.5 sec) for gamma 30-50 Hz band frequency. The EEG coherence was calculated as the normalized cross-gamma power spectrum per frequency of signals recorded in the frontal and parietal sites of the scalp. Results are demonstrated in FIGS. 13-14 demonstrate results before a training is performed; FIGS. 15-16 demonstrate results for before- and after-training was performed.

FIGS. 13A and 13B demonstrate the results of 12 participants (n=12, 6 young <40 and 6 old >60), which were presented with tasks having one, two or three items (colored shape/s), as demonstrated in FIG. 12. FIG. 13A demonstrated the Accuracy rate (between 0 and 1) and FIG. 13B demonstrates the response time in mSec. As expected, young participants showed superior response time and accuracy in memory test.

FIGS. 14A and 14B demonstrate the Gamma coherence, for the same tasks and participants as in the results of FIGS. 13A and 13B. The EEG readings analyzed Gamma Coherence was calculated between Frontal and Parietal sites (FIG. 14A) and between the two Parietal sites across hemispheres (FIG. 14B); where:
Working Memory (WM) load effect is the partial effect of the number of items on coherence.
Age effect is the partial effect of the age group.
The Linear Mixed Effects (LME) model is particularly useful when dealing with repeated measures and nested data; unlike 2-way ANOVA with two independent variables it doesn't assume equal variance across groups. It provides a more realistic representation of the underlying structure in the data and allows for the estimation of both fixed and random effects. It is an extension of the linear regression model that allows for the incorporation of both fixed effects and random effects.
Fixed Effects: These represent the population-level effects and are similar to the coefficients in a standard linear regression model. They capture the average relationship between the independent variables and the dependent variable across all levels.
Random Effects: These account for variability at different levels of the hierarchy. Unlike fixed effects, random effects are considered to be drawn from a larger population and are used to model the variability between different groups or clusters. Random effects are introduced to account for correlation and heterogeneity within groups.
   As demonstrated, the results show highly significant increase of coherence with memory load and a significant higher coherence for the young (age <40) vs. older (age >60) participants. Significance was assessed using the Linear Mixed effects Model (LME).

FIGS. 15A-15H and 16A-16F are related to the following experiments. In the experiments, blur visual training-tasks were used, also described in WO2023/073715A2. The training included a set of training sessions, of 10-15 minutes, per each session, and within each training session a collection of visual stimulation challenges, in which an 80 years-old (Subject No. 1) and a 67 year-old (Subject No. 2) were presented with a 1^{st} image and shortly thereafter with a 2^{nd} image. Both images (1^{st} & 2^{nd}) were identical, with a single difference: their blurriness degree; one of them had higher blur degree than the other. The users were asked, post the presentation of the two images, to specify, which one of the images contained a higher degree of blurriness. If a user provided a series of correct responses, the blur difference between the two images was reduced (so that it would be harder to detect), and the user was presented again with these challenges.

Prior to the training, the following parameters were measured for each user:
1. Accuracy of responses, in a working memory test (e.g., as demonstrated in FIG. 12);
2. The average time it takes for a user to respond, in the working memory test;
3. Coherence (strength of connectivity) of Gamma waves, between visual processing areas of the brain (P7, P8) and a cognitive area of the brain (Fz); and
4. P300 of the cognitive area (Fz).

During training, the frequency power (measured by dB of Gamma waves in the 30-49 Hz range) was measured in both the visual processing areas of the brain (P7, P8) and a cognitive area of the brain (Fz), during multiple visual task challenges, which included a presentation of the 1^{st} image and shortly thereafter the 2^{nd} image. Both images were defined to be with varying degrees of difference in blurriness, starting from 10% and going down to 2%. The frequency power was measured within 0.5 seconds from the presentation of 1^{st} image in the task (essentially allowing the information to be processed both by the visual processing points and the cognitive processing points—P7, P8 and Fz, respectively). Both images were randomly assigned to either the higher or lower degree of blurriness. The average frequency power responses, in both P7+P8 and Fz, were significantly elevated in response to a higher blur degree, as demonstrated in FIGS. 16E and 16F for the both (average) Subjects No. 1 and No. 2; Gamma Power (30-50 Hz) calculated 0.5 Sec after the first stimulus (memory time), baseline corrected to 0.5 Sec pre-stimulus.

Post, namely after the end of the training period, a re-evaluation of the following parameters was conducted for the users:
1. Accuracy of responses; in a working memory test;
2. The average time it takes for a user to respond, in the working memory test;
3. Coherence (strength of connectivity) of Gamma waves between visual processing areas of the brain (P7, P8) and a cognitive area of the brain (Fz); and
4. P300 of the cognitive area (Fz).

The training provided a multitude of improvements for the users. Cognitive abilities have increased and/or improved, as demonstrated in the higher accuracy of responses in the working memory test, while response time did not change materially. In addition, coherence (strength of connectivity) between visual processing areas of the brain (P7, P8) and a cognitive area of the brain (Fz) has substantially increased as well, as demonstrated in FIG. 15A for Subject No. 1 and FIG. 15C for Subject No. 2. In addition, training has a substantial positive effect on P300 as well, in both amplitude and speed as demonstrated in FIGS. 16A and 16B for Subject No. 1.

FIGS. 15A, 15B, 15C and 15D demonstrate the EEG readings analyzed Gamma coherence before-(line with circles) and after-(line with triangles) blur-task training sessions, of two subjects; an 80 years-old (Subject No. 1, as in FIGS. 15A and 15B) and a 67 years-old (Subject No. 2, as in FIGS. 15C and 15D). The EEG Gamma Coherence was calculated, as in the following figures, between Frontal and Parietal sites (FIGS. 15A and 15C) and between the two Parietal sites across hemispheres (FIGS. 15B and 15D). The results show higher coherence after training. FIGS. 15E, 15F, 15G and 15H, demonstrate the resulted improvement in their cognitive abilities, for performing working memory tests; FIGS. 15E and 15F demonstrate an increase and improvement in their accuracy, therefore in their cognitive abilities; and FIGS. 15G and 15H demonstrate their response time in [mSec].

FIGS. 16A and 16B demonstrate P300 (measured via frontal electrode, Fz) reaction difference, between a presentation of the target (simple-line) and non-target (dashed-line) during a WM task (similar to the task discussed in FIG. 12), before-(FIG. 16A) and after-(FIG. 16B) a training of the 80 years-old (Subject No. 1), blur-task training sessions The stimulus was provided at 2000 mSec and lasted for 500 mSec, thus the gray rectangle refers to the stimulus duration. FIGS. 16C and 16D demonstrate P300 magnitude (FIG. 16C) and P300 latency (FIG. 16D) pre- and post-training of the 80 years-old (Subject No. 1); same training and task as for the tests as of FIGS. 16A and 16B. As shown, the training increased the amplitude of P300 and shortened its latency.

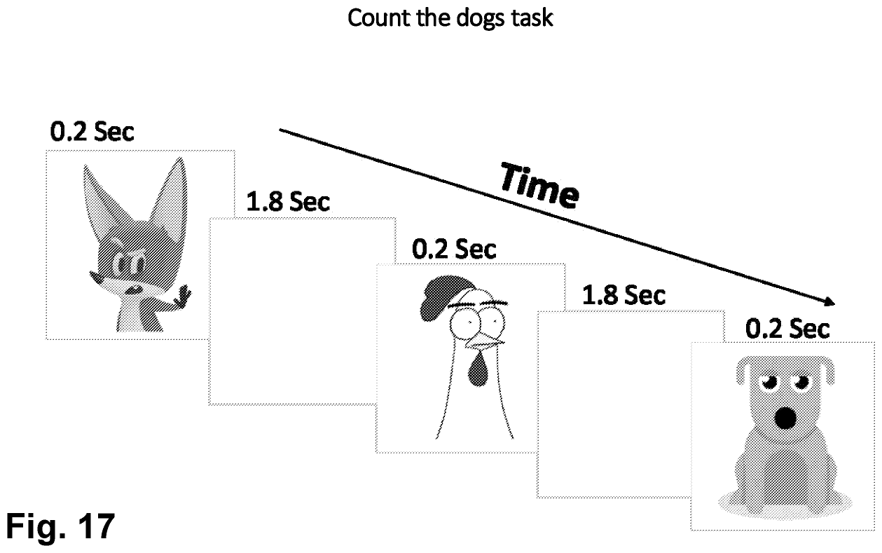

FIG. 17 demonstrates a “count the dogs” task. Twenty one (21) participants were presented with a slideshow of animal cartoon images (0.5 Hz) and were asked to report at the end of the task, the number of dogs that were presented to them (Target image).

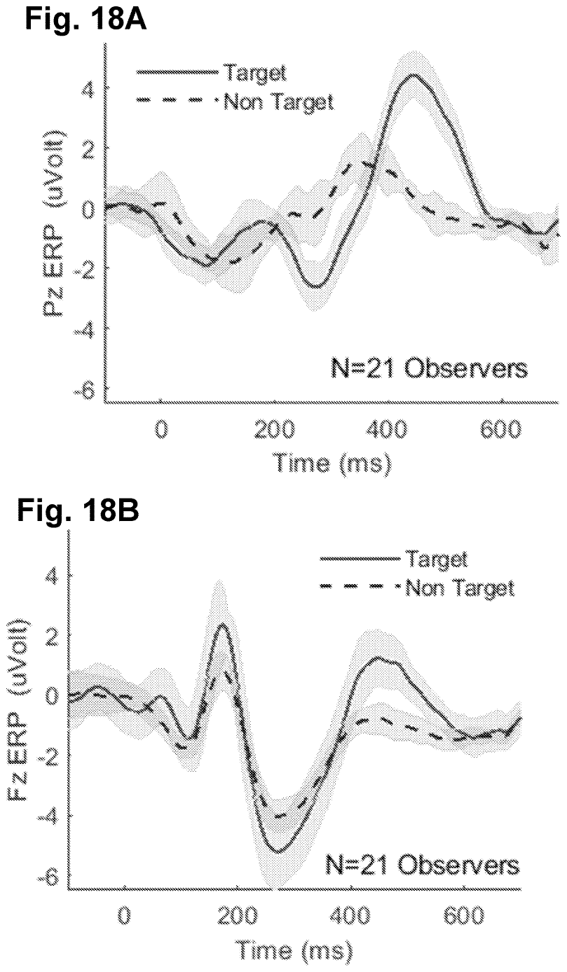

FIGS. 18A and 18B demonstrate the P300 response measured during the “count the dogs” task, as in FIG. 17, via Pz (FIG. 18A) and via Fz (FIG. 18B), during the presentation of the target (simple-line) and non-target (dashed-line). The results show that the P300 peak is larger for the target response then for the non-target response. The results are similar for the frontal (Fz) and parietal (Pz) electrodes.

Different embodiments are disclosed herein. Features of certain embodiments may be combined with features of other embodiments; thus, certain embodiments may be combinations of features of multiple embodiments.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A processor implemented method for increasing a user's cognitive abilities, the method comprising displaying to the user one or more visual training-tasks, via at least one session Sᵢ (S₁, S₂ . . . S_{M}), each session comprises a number (K_{S}) of time steps of K_{S} visual training-tasks, requiring the user's one or more responses; wherein the visual training-tasks are configured to induce an amplification of the brain's frequency power, in reaction to at least the provided visual training-tasks.

2. The method of claim 1, further comprising:
receiving and/or collecting, at a given time-step N (N=K, 1≤K≤K_{S}) of a session Sᵢ of K_{S} time steps, the user's response-profile to a displayed visual training-task, displayed at said given time step (N=K);
analyzing the user's response-profile, received at said given time-step (N=K) and optionally any former time-step/s (N<K) of said session Sᵢ;
repeating the step of displaying, at following time-step/s (K=K+1) of said session Sᵢ, a predetermined number of times K≤K_{S} for said session Sᵢ and/or until the response-profile has reached a predetermined threshold.

3. The method of claim 1, further comprising:
receiving and/or collecting, at a last time-step (N=K_{S}) of a session Sᵢ of time-steps (N=K, 1≤K≤K_{S}), the user's response-profile to a plurality of displayed visual training-tasks, displayed at time steps N (N=K, 1≤K≤K_{S}) of session Sᵢ;
analyzing the user's response-profile, received at said last time-step (N=K_{S}) of session Sᵢ;
repeating the steps of displaying, at following time-steps N (N=K, 1≤K≤K_{S}) of a following session Sᵢ, a predetermined number of sessions and/or until the response-profile has reached a predetermined threshold.

4. The method of claim 1, wherein inducement of the amplification of the brain's frequency power; comprises increasing of the power of the Gamma wave.

5. The method of claim 1, wherein inducement of the amplification of the brain's frequency power; comprises at least one of:
amplification of a P300 positive brainwave component; and
shortening of a P300 latency of the brain's reaction, after the user's response to the provided visual training-task.

6. The method of claim 1, wherein the visual training-task/s is/are selected to induce an amplification of the brain's frequency power in reaction at least one visual test-task and/or test-stimulation, which was/were not provided to said user by said method.

7. The method of claim 1 or 6, wherein the visual task comprises differentiating between displays of target and non-target images.

8. The method of claim 1, wherein the induced amplification of the brain's frequency power, in reaction to the provided visual training-tasks, is configured for improving a condition of a patient suffering from at least one of: sub-normal cognitive response, sub-normal motoric response, Alzheimer, depression, schizophrenia, ADHD, dyslexia, Parkinson, multiple sclerosis and any combination thereof.

9. The method of claim 1, further comprising a use in the treatment of at least one of: sub-normal cognitive response, sub-normal motoric response, Alzheimer, depression, schizophrenia, ADHD, dyslexia, Parkinson, multiple sclerosis and any combination thereof.

10. (canceled)

11. (canceled)

12. (canceled)

13. (canceled)

14. (canceled)

15. (canceled)

16. (canceled)

17. A processor implemented method for increasing a user's cognitive abilities, the method comprising displaying to the user one or more training stimulating-images, via at least one session (S₁, S₂ . . . S_{M}) each session comprises a number (K_{S}) of time steps of K_{S} training stimulating-images; wherein the training stimulating-images are configured to induce an amplification of the brain's frequency power, in reaction to at least to the provided training stimulating-images.

18. The method of claim 17, wherein inducement of the amplification of the brain's frequency power; comprises increasing of the power of the Gamma wave.

19. The method of claim 17, wherein inducement of the amplification of the brain's frequency power; comprises at least one of:
amplification of a P300 positive brainwave component; and
shortening of a P300 latency of the brain's reaction, after the displayed training stimulating-image.

20. The method of claim 17, wherein the training stimulating-image are selected to induce an amplification of the brain's frequency power in reaction at least test-image and/or test-visual-stimulation, which were not provided to said user by said method.

21. The method of claim 17, wherein the induced amplification of the brain's frequency power, in reaction to the provided visual-stimulation, is configured for improving a condition of a patient suffering from at least one of: sub-normal cognitive response, sub-normal motoric response, Alzheimer, depression, schizophrenia, ADHD, dyslexia, Parkinson, multiple sclerosis and any combination thereof.

22. The method of claim 17, further comprising a use in the treatment of at least one of: sub-normal cognitive response, sub-normal motoric response, Alzheimer, depression, schizophrenia, ADHD, dyslexia, Parkinson, multiple sclerosis and any combination thereof.

23. (canceled)

24. (canceled)

25. (canceled)

26. (canceled)

27. (canceled)

28. A device configured to present images to a user, comprising:
at least one processor configured to execute the method steps according to claim 1 and 17;
at least one display-device, configured to display the visual tasks and stimulating images to the user.

29. The device of claim 28, further comprising at least one input-device, configured to collect and interpret, at any given time-step (N=K), the user's response to the displayed images as in claims 2 and 3.

30. The device of claim 28, further comprising an apparatus selected from: a computer, a smart phone, a tablet and any combination thereof.

31. The device of claim 28, further comprising at least one of: data storage for said user's inputs and provided analyses, an input device, a speaker device, a microphone device, a computer mouse.
